(19) United States
(12) Patent Application Publication
(10) Pub. No.: US 2004/0105860 A1
(43) Pub. Date: Jun. 3, 2004

(54) Cell modulation using a cytoskeletal protein

(76) Inventors: Douglas W. Losordo, Winchester, MA (US); Raj Kishore, Nashua, NH (US)

Correspondence Address: EDWARDS & ANGELL, LLP, P.O. BOX 55874, BOSTON, MA, 02205, US

(21) Appl. No.: 10/633,407

(22) Filed: Aug. 1, 2003

Related U.S. Application Data
(60) Provisional application No. 60/400,084, filed on Aug. 1, 2002.

(51) Int. Cl. A61K 38/17; A61K 48/00; A61K 39/395

(52) U.S. Cl. 424/145.1; 514/44; 514/12

(57) ABSTRACT

Disclosed are compositions and methods for modulating endothelial cells (ECs) in a mammal. Practice of the invention generally involves changing activity of the ezrin cytoskeletal protein sufficient to increase or decrease proliferation of the cells. Also disclosed are useful screens for detecting agents capable of modulating ezrin activity. The invention has a variety of useful applications including use in the treatment of diseases associated with unsatisfactory EC proliferation.

### CROSS-REFERENCE TO RELATED APPLICATION

The present application is a continuation of U.S. Provisional Application No: 60/400,084 as filed on Aug. 1, 2002, the disclosure of which is incorporated herein by reference.

### STATEMENT OF GOVERNMENT INTEREST

Funding for the present invention was provided in part by the Government of the United States by virtue of grant HL63414 (ROI) from the National Institutes of Health. Accordingly, the Government of the United States has certain rights in and to the invention claimed herein.

### FIELD OF THE INVENTION

The invention relates to compositions and methods for modulating endothelial cell (EC) and/or endothelial progenitor cell (EPC) function in a mammal. Practice of the invention generally involves modulating activity of the ezrin cytoskeletal protein sufficient to increase or decrease proliferation and/or differentiation of the cells. Further disclosed are useful screens for detecting agents capable of modulating the ezrin activity. The invention has a variety of useful applications including use in the treatment of medical indications associated with unsatisfactory EC proliferation.

### BACKGROUND

There is nearly universal recognition that blood vessels help supply oxygen and nutrients to living tissues. Blood vessels also facilitate removal of waste products. Blood vessels are renewed by a process termed “angiogenesis”. See generally Folkman and Shing, J. Biol. Chem. 267 (16), 10931-10934 (1992).

Angiogenesis is understood to be important for the well-being of most mammals. As an illustration, it has been disclosed as being an essential process for facilitating reproduction, wound repair and development.

Much attention has focused on understanding how angiogeneis is regulated. The process is thought to begin with the degradation of the basement membrane by proteases secreted from endothelial cells (EC). Cell migration, proliferation and vascular loop formation are also thought to have important roles. What has been referred to as endothelial cell progenitors (EPC) are thought to give rise to the EC. See eg., WO 99/45775 by Isner, J. et al. and references cited therein.

Insufficient angiogenesis is thought to occur in conditions such as ulcers, strokes, and heart attacks. Restenosis following surgical manipulations such as balloon angioplasty has been particularly problematic. See Krasinski, K. et al. (2001) Circulation 104:1754-1756 (2001) and references cited therein.

Too much angiogenesis also presents difficulties. For instance, there is almost universal recognition that the development of certain tumors and cysts are greatly assisted by uncontrolled angiogenesis. Certain vasculopathies such as atherosclerosis may also benefit.

The concept of administering therapeutic compositions to facilitate new vascularization has long been recognized. For instance, gene therapy approaches have received increasing acceptance and use. See generally Takeshita, et al., Circulation, 90:228-234 (1994); Takeshita, et al., J Clin Invest, 93:662-70 (1994); Tsurumi et al., Circulation, 94(12):3281-3290 (1996) and Isner et al. Lancet 348:370 (1996).

Tumor necrosis factor (TNF) has been reported to be a cytokine with many important functions. See Rosenkranz-Weiss P, et al. J Clin Invest. 1994;93:2236-43; Wang P, et al. Am J Physiol. 1994;266:H2535-41; and references cited therein.

For example, there are reports that TNF is expressed in arteries during atherosclerosis and restenosis with increased expression after balloon injury in multiple animal models. See eg., Kaartinen M, et al. Circulation. 1996;94:2787-92; and Tanaka H, et al. Arterioscler Thromb VascBiol. 1996;16:12-8.

It has been disclosed that blocking TNF improves re-endothelialization after balloon angioplasty. Moreover, in vitro exposure of primary ECs to TNF has been reported to inhibit proliferation and enhance apoptosis. See Krasinski, K. et al. (2001), supra.

There have been efforts to understand how TNF impacts cell function. For example, it has been reported that TNF mediates EC cell cycle arrest. Cell cycle regulatory genes, including cyclin A, are thought to play a significant role. See Krasinski, K. et al. (2001), supra.

Cyclin A gene mRNA levels are thought to increase in S phase. The promoter of this gene is believed to harbor regulatory elements that facilitate cell cycle control and transcription of the gene. The transcriptional control elements are thought to include two repressor binding sites: a cell cycle dependent element (CDE) and a cell cycle gene homology region (CHR). See Beutler B. J Investig Med. 1995;43:227-35; Liu, N. et al. Nucleic Acids Res. 1997;25 :4915-20; and references cited therein.

A cellular factor called CDF-1 is believed to bind the CDE and CHR element and repress cyclin A expression. Another factor, CHF, has been reported to bind specifically to the cyclin A CHR. See Schulze, A. et al. PNAS (USA) 92:11264-11268; and Philips, A. et al. (1999) Oncogene 18:6222-32.

There has been attempts to understand the cytoskeleton. It has been described as a filamentous web of actin, microtubules, intermediate filaments and associated proteins. The cytoskeleton is generally positioned in the cell between the outer membrane and the nucleus. See generally, Lodish, H. et al. (2000) in Molecular Cell Biology 4^{th} Ed. W. H Freeman (New York); and references disclosed therein.

Several cytoskeleton associated proteins have been described. These include ezrin, radixin, moesin (collectively referred to as ERM proteins ); and merlin (moesin-ezrin-radixin-like protein). Nucleotide and amino acid sequence information has been disclosed for these proteins. See e.g., Turunen, O. et al., J. Biol. Chem., 264: pp.16727-16732, 1989; Funayama, N. et al., J. Cell Biol., vol. 115, p. 1039-1048, 1991; Lankes, W. T. et al., PNAS (USA) pp. 8297-8301, 1991; Gould et al. EMBO J. 8:4133-4142 (1989) and Lodish, H., supra.

See also U.S. Pat. Nos. 5,773,573; 6,399,584; 6,225,442 and references cited therein for additional information.

It would be desirable to have methods of using a cytoskeletal protein or nucleic acid encoding same to modulate cells. It would be more desirable to have methods that use ezrin or ezrin encoding nucleic acid to increase or decrease proliferation of the endothelial cells, particularly to modulate vascularization in a mammal such as a human patient.

### SUMMARY OF THE INVENTION

The invention generally relates to use of a cytoskeletal protein and agents that modify same to modulate cells. More specifically, the invention relates to methods of modulating endothelial cells (ECs), endothelial cell precursors (EPCs) and related cells by increasing or decreasing activity of the ezrin cytoskeletal protein. The invention has a broad spectrum of important applications including use to modulate vascularization in a mammal and especially a human patient.

We have discovered that it possible to modulate cells by changing activity of the ezrin protein. More specifically, we have found that under certain conditions ezrin associates with nucleic acid to impact cell proliferation. Without wishing to be bound to theory, it is believed that ezrin is a new nucleic acid binding protein that can modulate transcription of the cyclin A gene: a key cell cycle regulator. For instance, when bound to the cyclin A gene, we believe ezrin exerts a substantial effect on cell cycling and proliferation. Our discovery is surprising in view of past reports suggesting that ezrin is primarily a structural protein of the cytoskeleton. Accordingly, the invention has a broad spectrum of important applications including use in settings where modulation of cell cycling and/or proliferation is needed in vitro and in vivo. Further uses of the invention include preventing, treating and/or reducing the severity of a variety of medical conditions associated with undesired cell proliferation. The invention further provides new screens that can be employed to detect molecules that modulate ezrin activity.

Thus we have uncovered an important link between the cytoskeleton, gene transcription and proliferative cues that can be used in accord with the invention to prevent or treat disease, or to help detect agents that can regulate the proliferation and/or differentiation of EC, EPC and other cell types.

More specifically, we have unexpectedly found that when ezrin is bound to the cyclin A gene, expression from that gene is decreased. Cell cycling and proliferation is hindered. Similarly, when the cyclin A gene is not so bound by the ezrin protein, expression from the cyclin A gene is increased and cell cycling and proliferation is enhanced. Practice of the invention takes advantage of these discoveries by providing new therapies and screening strategies that involve manipulation of the ezrin protein, specifically by modulating ezrin binding to the cyclin A gene.

Accordingly, and in one aspect, the invention provides a method for modulating cell function in a mammal that involves increasing or decreasing ezrin activity by an amount sufficient to modulate proliferation of the cells. Typical methods further include administering to the mammal at least one ezrin modulating agent sufficient to modulate (increase or decrease) ezrin activity and modulate cell proliferation. It will be appreciated that the type of cell modulated by the invention will depend in large part on intended use. However for many embodiments, preferred cells will be endothelial cells (EC) and endothelial progenitor cells (EPCs). The invention thus provides in one embodiment a method for modulating ECs and/or EPCs that involves increasing or decreasing ezrin activity by an amount sufficient to modulate cell proliferation.

The present invention has many important uses and advantages. For instance, it can be employed to increase ezrin DNA binding activity in cells, thereby decreasing cell proliferation. That is, by enhancing ezrin binding to the cyclin A gene (or an effective portion thereof), the invention can be used to reduce or in some cases block cyclin A expression. Cell cycle entry is delayed and proliferation is decreased or in some cases blocked. In embodiments in which increased cell proliferation is desired, the invention provides a method for decreasing the DNA binding of ezrin to the cyclin A gene to provide a controlled increase in cyclin A gene expression. In this invention example, cell cycling is facilitated and cell proliferation is increased by the decrease in ezrin binding to the cyclin A gene or the portion thereof. As defined herein, “an effective portion” of the cyclin A gene will include one or more genetic components necessary and/or sufficient to express the gene. Preferably, one of the genetic components is a mammalian cyclin A promoter element as described herein.

The invention also provides a method for inducing the formation of new blood vessels in a mammal and particularly a human patient. In one embodiment, the method includes decreasing the DNA binding activity of the protein in an amount sufficient to increase formation of the new blood vessels in the mammal. Alternatively, the invention can be used to prevent or totally eliminate formation of new blood vessels by increasing the ezrin DNA binding activity in an amount sufficient to decrease formation of the new vessels. In both embodiments, the invention provides a new method of controlling vascularization in vitro and in vivo by taking advantage of a previously undiscovered DNA binding feature of the ezrin protein. Typically, such methods further include administering to the mammal at least one ezrin modulating agent sufficient to increase or decrease the DNA binding activity of the ezrin protein relative to a suitable control.

Also provided is a method for reducing the severity of blood vessel damage in a mammal and particularly a human patient. In one embodiment, that method includes decreasing the ezrin DNA binding activity in ECs, EPCs or both, prior to and/or during a time the mammal is exposed to conditions conducive to damaging the blood vessels. Alternatively, or at the same time, the invention can be utilized to decrease the ezrin activity in the EC and EPC cells eg., to provide a ready source of proliferation competent ECs. Preferably, the decrease in ezrin activity is sufficient to reduce the severity of the blood vessel damage in the mammal. Typically, such methods further include the step of administering to the mammal at least one ezrin modulating agent in an amount sufficient to increase or decrease ezrin DNA binding activity relative to a control.

Accordingly, the invention provides in one embodiment a convenient ex vivo approach for enhancing numbers of isolated EPCs. That is, it provides a method of enhancing EPC production ex vivo or in vitro by decreasing ezrin DNA binding activity in the cells sufficient to augment cell proliferation.

Alternatively, practice of the invention can be used to reduce or block angiogenesis in a mammal preferably by increasing the ezrin DNA binding activity in EC and/or the EPC sufficient to decrease the angiogenesis. Typical methods involve administering to the mammal at least one ezrin modulating agent sufficient to decrease the ezrin DNA binding activity relative to a suitable control.

Further provided are simple testing methods that can be used to detect compounds that modulate ezrin activity e.g., by at least about 10% or more relative to a suitable control (eg., saline or buffer), preferably at least about 25% relative to that control. Accordingly, the invention also relates to methods for detecting and analyzing compounds that increase or decrease ezrin DNA binding activity and preferably also demonstrate therapeutic capacity to modulate vascularization of endothelial cells (EC and/or EPC). Preferred detection and analysis platforms include both in vitro and in vivo assays to determine therapeutic capacity to modulate the ezrin activity.

A suitable detection assay according to the invention generally tests binding between the cyclin A gene or an effective portion thereof and ezrin (or an effective DNA binding fragment or derivative of ezrin ). Such assays are typically conducted in the presence of at least one candidate compound. Such tests can be conducted in vitro or in vivo as needed and may be carried-out according to one or a combination of specific assay formats.

A particular in vitro assay involves at least one of and preferably all of the following steps:

1) introducing into cells at least one type of nucleic acid that includes at least an effective portion of a mammalian cyclin A gene up to the entire gene which gene is optionally and operably linked to sequence encoding at least one detectable label,

2) adding at least one known or candidate ezrin modulating compound to the cells (eg., TNF-α or an effective fragment thereof),

3) culturing the cells under conditions suited to express the nucleic acid and measuring the detectably labeled sequence in the presence and absence of the compound; and

4) determining the effect of the compound on the cells such as by detecting and optionally measuring the detectable label. If desired, the method can further include measuring at least one of cell proliferation and cell cycling in accord with conventional techniques.

A particular label for use with the method is a detectable amino acid sequence that is preferably expressed in-frame with the mammalian cyclin A protein or portion of the mammlian cyclin A gene encoding same eg., a promoter element. Preferred labels may be directly or indirectly detectable. More particular labels will be well-suited for automated or semi-automated analysis of pools of candidate compounds. In this regard, colored, fluorescent, chemiluminescent or phosphorescent labels expressed in-frame with respect to the cyclin A gene (or effective fragment thereof) will be preferred. Such an assay can effectively measure capacity of the candidate compound to increase or decrease the capacity of the ezrin protein to bind the cyclin A gene and shutdown or reduce cyclin A gene expression. Illustrative cells for use with the method include, but are not limited to, EC and EPC cells.

Another particular assay of the invention detects and optionally measures DNA binding between ezrin (or a DNA binding fragment or a derivative thereof) and at least an effective portion of the mammalian cyclin A gene. A preferred portion of the cyclin A gene for use in the assay is the transcriptional promoter of that gene which promoter will include some or all of the specific elements described herein. For example, and in one embodiment, the assay includes at least one and preferably all of the following steps:

1) incubating an effective portion of the mammalian cyclin A gene (eg., a single-stranded nucleic acid fragment including at least part of the transcriptional promoter of the gene) with the ezrin protein (or DNA binding fragment or derivative thereof), wherein the incubation is conducted under conditions sufficient to form a specific binding pair between the cyclin A gene (or effective portion) and the ezrin protein (or effective fragment or derivative),

2) adding at least one known or candidate ezrin modulating compound (eg., TNF-alpha) to the incubation medium; and

3) detecting presence of a specific binding pair between the cyclin A gene (or fragment) and the ezrin protein (or fragment or derivative) in the presence and absence of the compound, wherein a reduction or absence of the binding pair is taken to be indicative of a compound that reduces or blocks ezrin binding to the cyclin A gene. Alternatively, an increase in the formation or stability of the binding pair can be taken as being indicative of a compound that enhances ezrin binding to the cyclin A gene.

The in vitro assays of the invention are flexible and can be adapted to suit an intended use.

For instance, the methods can be used as a general screening platform to test large populations of candidate molecules. If desired, cells can be synchronized by standard methods (eg., serum deprivation). If desired, identified compounds can be further screened in the DNA binding assay described above which in particular embodiments can include use of purified (or semi-purified) assay components such as cell lysates or extracts. This screening strategy has the advantage of identifying ezrin modulating compounds from potentially large pools or libraries of candidate compounds. Of course, optimal use of the invention is not tied to a particular testing regimen so long as intended screening results are obtained.

The invention also features a method for detecting a compound that modulates the DNA binding activity of ezrin. In one embodiment, the method includes at least one and preferably all of the following steps:

1) adding at least one known or candidate ezrin modulating compound to the cells,

2) culturing the cells under conditions suited to increase or decrease ezrin phosphorylation relative to a control; and

3) identifying an increase or decrease in ezrin phosphorylation (eg., tyrosine phosphorylation) relative to a suitable control as being indicative of the compound.

Further provided is a pharmaceutical product for inducing neovascularization in a mammal. In one embodiment ,the product comprises endothelial cells and the product includes at least one ezrin modulating agent. Preferably, the cells are formulated to be physiologically acceptable to a mammal.

The invention also provides a kit for the introduction of a endothelial cells into a mammal. In one embodiment, the kit includes at least one ezrin modulating agent and optionally at least one angiogenic or hematopoietic protein or nucleic acid encoding same. Preferred kits further include a pharmacologically acceptable carrier solution, nucleic acid or mitogen, means for delivering the cells and directions for using the kit.

Other features and advantages of the invention are described below.

### BRIEF DESCRIPTION OF THE DRAWINGS

FIGS. 1 A-D show that Tumor Necrosis Factor (TNF) suppresses cyclin A mRNA expression. FIG. 1A is a representation of an authoradiograph showing that TNF suppresses cyclin A mRNA expression. FIGS. 1B-D are graphs showing mRNA expression of cyclin A (FIG. 1B), cyclin B (FIG. 1c) and cyclin D1 (FIG. 1D)

FIGS. 2A-B show that de novo transcription of cyclin A mRNA is blunted by TNF. FIG. 2A is a representation of an autoradiograph in which nuclei harvested from quiescent and serum stimulated EC cultured with or without TNF were subjected to nuclear run-on analysis. Data from 3 similar experiments was quantified by densitometric analysis in FIG. 2B.

FIGS. 3A-B show that TNF destabilizes cyclin A mRNA. FIG. 3A is a representation of an autoradiograph in which total RNA from ECs treated or not with TNF in the presence or absence of actinomycin D was hybridized with in vitro transcribed antisense cyclin probe and analyzed by RPA. FIG. 3B is a graph representing quantified values for % remaining cyclin A and cyclin D1 mRNA at indicated time points.

FIG. 4 is a diagram showing CDE-CHR cis-elements are essential for efficient cyclin A promoter activity.

FIG. 5 is a diagram showing TNF mediated suppression of cyclin A promoter activity requires CDE-CHR elements.

FIG. 6 is a representation of an autoradiograph showing that CDE-CHR DNA binding activity is modulated by TNF.

FIG. 7 is a representation of an autoradiograph showing that TNF increases the binding activity to the CHR elements.

FIGS. 8A-E are representations of autoradiographs showing specific binding of TNF induced 84 kDa protein to the CHR elements.

FIGS. 9A-D are gel representations showing that cyclin A CHR-binding protein is ezrin.

FIG. 10A is a gel representation and FIGS. 10B-C are photographs of cultured cells showing that TNF up-regulates and translocates ezrin.

FIGS. 11A-C are photographs of cultured cells treated with control (empty vector) or vector encoding wild-type (wt) or dominant negative (dn) ezrin; and FIGS. 11D-E are graphs showing that ezrin mediates TNF-induced inhibition of EC proliferation.

FIG. 12A is a representation of a crosslinking experiment showing ezrin binding to the cyclin A promoter, FIG. 12B is a graph and FIG. 12C is a gel representation showing that ezrin binds to cyclin A promoter in vivo and dominant negative ezrin attenuates TNF-induced cyclin A down-regulation.

FIGS. 13A and B are photographs of cells transfected with wtEzrin (13A) or dnEzrin (13B), FIGS. 13C-D are photographs of mouse hindlimbs and FIG. 13C is a graph showing that dominant negative ezrin transfected HUVEC facilitates angiogenesis in ischemic hind limb.

FIGS. 14A-C are gel representations showing that TNF-induced RhoA Kinase phosphorylates ezrin.

FIGS. 15A-C are graphs showing that inhibition of RhoA kinase partially attenuates TNF-mediated inhibitory effects on EC proliferation.

### DETAILED DESCRIPTION OF THE INVENTION

As discussed, the present invention relates to use of a cytoskeletal protein to modulate cells. Particular cells of interest are involved in vascularization such as endothelial cells (ECs) and endothelial progenitor cells (EPCs). More specifically, the invention relates to methods of modulating ECs and EPCs by increasing or decreasing the DNA binding activity of the ezrin cytoskeletal protein. The invention has a broad spectrum of important uses including providing important screens to detect compounds that can modulate vascularization via an increase or decrease ezrin binding to the cyclin A gene.

It has been unexpectedly found that the cytoskeletal protein ezrin is a DNA binding molecule. More particularly, it has been discovered that ezrin binds to the promoter element of the cyclin A gene; an acknowledged regulator of cell cycling. That is, changes in the intracellular level of ezrin (particularly in the nucleus) is believed to impact cyclin A gene function and alter cell cycling. Accordingly, it is believed that ezrin has potential to regulate a wide variety of cell functions including the cell proliferation that typically ensues along with cell cycling.

There is recognition that cyclin A promotes cell cycle progression by associating with and stimulating cyclin dependent kinases cdc2 and cdk2. It has been disclosed as being a critical regulator of the cell cycle at both G1-S junction and during G2-M transition²⁸. There is further understanding that the level of cyclin A is controlled primarily by cyclical changes in mRNA expression^{12, 28, 29}. There is growing acknowledgement that cytokines such as TGFβ1, INFγ and TNF also inhibit the expression of cyclin A in various cell types^{13, 22, 23}.

Transcriptional repression of cyclin A has been reported in response to both TGFβ1 and INFγ^{13, 22}. Inhibition of cyclin A promoter activity by TGFβ1 is thought to require an intact ATF site, and this effect involves decreased phosphorylation of CREB and ATF-1^{22, 23}. INFγ mediated inhibition of cyclin A gene transcription is reported to be independent of individual cis-acting elements¹³. However, there is belief that neither the cis-elements that are the target of TNF nor the molecular mechanisms for TNF-mediated cyclin A transcriptional repression.

As discussed, the invention features a method for modulating (increasing or decreasing) cell cycling and particularly cell proliferation. Preferred cells include endothelial progenitor cell (EPC) and endothelial cells (EC). In one embodiment, the method includes decreasing ezrin DNA binding activity in an amount sufficient to enhance proliferation of the cells. Such ezrin activity can be modulated by one or a combination of strategies according to this invention.

Methods for detecting increases or decreases in endothelial cell proliferation are well known in the field and include visualization of sprouting in vivo, blood vessel length assays, cornea micropocket assays, conventional cell culture techniques including cell counting, ect. See eg., WO 99/45775 by Isner, J. et al. and references cited therein.

A variety of methods for identifying, making and using EC and EPC cells have been disclosed. See eg., the WO 99/45775 PCT application and U.S. Pat. Nos. 5,980,887; 6,258,787; and 6,121,246.

The present invention shows for the first time that it is possible to modulate cyclin A gene expression by modulating the DNA binding activity of ezrin. In this regard, it has been found that tumor necrosis factor (TNF), for instance, also exerts an inhibitory effect on cyclin A gene expression. That inhibition is believed to be mediated both by a transcriptional repression as well as by a reduction in mRNA stability. It is shown for the first time that CHR elements within cyclin A promoter are the target of TNF. That target is the DNA binding site of the ezrin protein, a fuinctionally novel, TNF inducible, 84 kDa protein which binds specifically to CHR elements and is involved in transcriptional repression of cyclin A.

Sequence information for the human cyclin A gene has been disclosed. See Liu, N et al. (1998) Oncogene 16:2957; Pines, J. (1993) Trends Biochem. Sci. 18:195-197; Schulze, A. et al. (1995) PNAS (USA) 92:11264 and references disclosed therein. See also product literature available from the Pharmingen company. See also reference nos. 21, 22, and 28 shown below.

Specifically preferred human cyclin A genes in accord with the invention include at least the CHR promoter sequence and typically also the CDE-CHR promoter sequence as provided in the Examples. It will also be understood that by “promoter” is meant a segment of DNA to which a transcriptional enzyme complex binds prior to initiating transcription of the gene.

With respect to preferred EC cells, TNF has been reported to activate inflammatory responses and influence angiogenesis. Other reports disclose that it may negatively affects EC proliferation and apoptosis^{9, 26, 27}. Inhibition of EC proliferation and enhanced apoptosis in response to TNF has significant bearing on endothelial recovery eg., following balloon angioplasty, which may in turn contribute to the development of restenosis.

Indeed, there is recognition that blockade of locally expressed TNF at the site of balloon injury improves the process of re-endothelialization⁸. There have been reports that in vitro exposure of primary EC to TNF inhibits their proliferation and impairs their ability to progress through the cell cycle^{9, 10,15}. Without wishing to be bound to theory, this inhibitory effect of TNF, at least partially, is believe to depend on the down-regulation of cell cycle regulatory genes, including that of cyclin A^{9, 15}. TNF-mediated repression of cyclin A has been previously reported in EC.

As discussed, the invention provides a method for modulating EC and/or EPC proliferation in a mammal that in one embodiment includes increasing or decreasing ezrin activity in the mammal by an amount sufficient to modulate proliferation of the cells. By the phrase “ezrin activity” is meant capacity to bind nucleic acid, particularly the mammalian cyclin A gene (or at least an effective fragment thereof), preferably the human cyclin A gene, as determined by at least one of the in vitro or in vivo assays described herein. In one invention embodiment, the ezrin activity is decreased by an amount sufficient to enhance the proliferation of the EC and/or EPC cells in the mammal.

In a more particular embodiment, the method further involves administering to the mammal at least one ezrin modulating agent sufficient to decrease the ezrin activity and enhance the EC proliferation. By the phrase “ezrin modulating agent” is meant a compound with capacity to increase or decrease ezrin activity relative to a suitable control as determined by at least one of the in vitro or in vivo assays described herein. Such agents can be small molecules (ic. having a molecular weight of less than about 1 OOOD) or amino acid sequences such as proteins, glycoproteins, antibodies, receptors, nucleic acids (DNA or RNA) as well as effective fragments or derivatives thereof that can increase or decrease ezrin activity as determined by one or more of the assays.

More preferred ezrin modulating agents include TNF-α as well as biological competitors (antagonists) thereof such as TNF soluble receptor protein (TNFsr), anti-TNF antibody; or an effective fragment or derivative thereof. Disclosure relating to the TNF superfamily can be found in Ware,C. et al. (1998) in The Cytokine Handbook, 3^{rd}. Ed., Thomson, A. W. ed., Academic Press (San Diego, Calif.), particularly pp. 549-592; Wajant, H. et al. (1999) Cytokine Growth Fact. Rev. 10:15-26; and Aggarwal, B and Reddy, S. (1994) in Guidebook to Cytokines and Their Receptors, Nicola, N Ed. Oxford Press (New York, N.Y.). A variety of TNF-α, TNFsr and anti-TNF antibody preparations are available from commercial suppliers such as Sigma-Aldrich (P.O. Box 14508, St. Louis, Mo. (USA)). See also the Examples section.

By the phrase “effective fragment” as it relates to an ezrin modulating agent is meant a portion of the agent (such as a protein, glycoprotein, nucleic acid (RNA or DNA), etc.) that has at least about 80% of the activity of the corresponding full-length agent in at least one of the assays described herein, preferably at least about 90% or 95% of that activity. By way of illustration and not limitation, an effective fragment of the anti-TF antibody would include fragments that specifically bind TNF-α eg., Fab, F(v), Fab′, F(ab′)₂ fragments, “half molecules” derived by reducing the disulfide bonds of immunoglobulins, single chain immunoglobulins, or other suitable antigen binding fragments (see e.g., Bird et al., Science, pp. 242-424 (1988); Huston et al., PNAS, (USA), 85:5879 (1988); Webber et al., Mol. Immunol., 32:249 (1995)).

By “derivative” of an ezrin modulating agent is meant an analogue having substantial identity to the agent. In embodiments in which the agent is a protein or nucleic acid sequence, for instance, the analogue is preferably at least about 90% identical to the agent as determined eg., by inspection or with the aid of a suitable computer program such as BLAST, FASTA or related programs, preferably at least about 95% identical. Suitable analogues include protein sequences having one or more conservative amino acid substitutions with respect to the corresponding agent. By “conservative” amino acid substitution is replacement of one amino acid residue for another having similar chemical properties (eg., replacing tyrosine with phenylalanine). Additionally suitable derivatives of the ezrin modulating modulating agents can have one or more deletions or insertions of amino acid or nucleic acid sequence provided such changes do not impact function as determined by one or more of the assays disclosed herein.

Particular TNF-α antagonists in accord with the invention (including TNF-α receptors and ligand binding fragments thereof) have been disclosed. See eg., U.S. Pat. Nos. 6,107,273; 6,015,557 and 5,605,690.

It has been reported that the Rho family protein include RhoA, RhoB, RhoC, Rac1, Rac2 and Cdc42. The Rho proteins share more than 50% sequence identity with each other and are thought to induce formation of stress fibers and focal contacts in response to extracellular signals. See eg., A. J. Ridley & A. Hall, Cell, 70, 389-399 (1992); and A. J. Ridley & A. Hall, EMBO J., 1353, 2600-2610 (1994)). The subfamily Rho is also considered to be implicated in physiological functions associated with cytoskeletal rearrangements, such as cell morphological change (H. F. Parterson et al., J. Cell Biol., 111, 1001-1007 (1990)), cell adhesion (Morii, N. et al., J. Biol. Chem., 267, 20921-20926 (1992); and Nusrat, A. et al., Proc. Natl. Acad. Sci. USA, 92, 10629-10633 (1995); cell motility (K. Takaishi et al., Oncogene, 9, 273-279 (1994), and cytokinesis (K. Kishi et al., J. Cell Biol., 120, 1187-1195 (1993)).

A variety of Rho kinase inhibitors have been disclosed. See eg., U.S. Pat. Nos. 6,451,825 and 6,218,410. See also Japanese Patent Unexamined Publication No. 62-89679, Japanese Patent Unexamined Publication No. 3-218356, Japanese Patent Unexamined Publication No. 4-273821, Japanese Patent Unexamined Publication No. 5-194401, Japanese Patent Unexamined Publication No. 6-41080 and WO95/28387. A preferred Rho A kinase inhibitor is Y27632 as discussed in the Examples below. The inhibitor can be obtained from commercial sources such as CalBiochem, Inc. (San Diego, Calif.).

In one method of the invention, the DNA binding activity of ezrin is decreased by contacting suitable ECs or EPCs with an effective amount of a competitor of TNF eg., TNF-α or a functional fragment thereof. Preferred examples of such competitors are known in the field and include, but are not limited to, TNF soluble receptor protein (TNFsr), an antagonist of TNF (sometimes referred to herein as a “TNF blocker”), and an anti-TNF antibody. See Krasinski, K. et al. (2001) Circulation 104:1754-1756 (2001) and references cited therein for disclosure relating to TNFsr. See also U.S. Pat. Nos. 6,416,757; 6,379,666; 6,107,273; and 6,015,557 (further disclosing a variety of TNF blockers and anti-TNF antibodies). Also envisioned are fragments or derivatives of TNFsr, TNF blocker (antagonist), and an anti-TNF antibody that decrease the DNA binding activity of ezrin. A preferred TNF competitor according to the invention reduces or blocks ezrin binding as determined by the standard cyclin A promoter binding assay described below.

Ezrin has attracted a significant level of interest and much is known about the structure and function of the cytoskelatal protein. The nucleic acid and amino acid sequence of the protein have been reported. See e.g., Turunen, O. et al., supra; Funayama, N. et al., supra; Lankes, W. T. et al., PNAS (USA) supra; Gould et al. EMBO J. supra; and U.S. Pat. Nos. 5,773,573; 6,399,584; and 6,225,442.

The invention can be practiced with a wide variety of suitable mammalian ezrin sequences, particularly those of rodent (mouse) and human origin. An illustration is a human ezrin reported as GenBank accession number P15311 and shown below in Table 1:

```
TABLE 1
  1 mpkpinvrvt tmdaelefai qpnttgkqlf dqvvktiglr evwyfglhyv dnkgfptwlk
  61 ldkkvsaqev rkenplqfkf rakfypedva eeliqditqk lfflqvkegi lsdeiycppe
121 tavllgsyav qakfgdynke vhksgylsse rlipqrvmdq hkltrdqwed riqvwhaehr
181 gmlkdnamle ylkiaqdlem yginyfeikn kkgtdlwlgv dalglniyek ddkltpkigf
241 pwseirnisf ndkkfvikpi dkkapdfvfy aprlrinkri lqlcmgnhel ymrrrkpdti
301 evqqmkaqar eekhqkqler qqletekkrr etverekeqm mrekeelmlr lqdyeektkk
361 aerelseqiq ralqleeerk raqeeaerle adrmaalrak eelerqavdq iksqeqlaae
421 laeytakial leearrrked eveewqhrak eaqddlvktk eelhlvmtap ppppppvyep
481 vsyhvqeslq degaeptgys aelssegird drneekrite aeknervqrq lvtlsselsq
541 ardenkrthn diihnenmrq grdkyktlrq irqgntkqri defeal
```

Suitable nucleic acids for use with the invention include those that encode at least part of a mammalian ezrin such as human ezrin. An example of an appropriate nucleic acid is the nucleic acid shown as GenBank accession number BC013903 as well as effective fragments thereof.

According to another approach, the ezrin activity is decreased by introducing, into the cells, an ezrin anti-sense nucleic acid. Methods for making and using a wide variety of anti-sense molecules have already been disclosed. See eg., U.S. Pat. No. 6,399,377 and references disclosed therein. Alternatively, or concomitantly, the ezrin activity can be decreased by introducing into the cells an anti-ezrin antibody or effective fragment thereof as known in the field.

Further ezrin modulating agents in accord with the invention include those dominantly and negatively acting fragments of mammalian ezrin including the human protein (sometimes referred to herein as dnEzrin). Preferred are ezrin protein fragments that lack at least about 10 amino acids of the wild-type (ie. normal 586 amino acid) ezrin protein, preferably at least about 100 amino acids, more preferably at least about 200 to about 300 amino acids, and even more preferably between about 400 to about 500 amino acids. Specifically preferred of such ezrin fragments lack amino acid sequence beginning from about the C-terminus of the ezrin protein. Even more preferred are ezrin fragments that include at least the first 300 amino acids from the N-terminus of the ezrin protein, preferably at least about the first 200 amino acids, and more preferably at least about the first 100 to about 125 amino acids with about the first 115 amino acids being preferred for many applications. Specific disclosure relating to such ezrin fragments has been disclosed. See eg., Andreoli, M M et al. (1995) J. Cell Biol. 128:1081; Algrain M. et al. (1993) J. Cell Biol. 120:129; and references cited therein. See also the Examples section.

Additionally suitable ezrin modulating agents include nucleic acids that encode dnEzrin including fragments and derivatives thereof.

Additionally preferred dominantly and negatively acting fragments of the mammalian ezrin protein significantly decrease ezrin activity relative to a control as determined by at least one of the assays disclosed herein.

Thus in certain invention embodiments, it will be useful to administer one or a combination of ezrin modulating agents that include or consist of nucleic acid such as those encoding dnEzrin as mentioned previously. Methods for administering a nucleic acid to a mammal and particularly direct injection to or near particular organs or tissue of interest (eg., heart) has been disclosed. See e.g., U.S. Pat. Nos. 5,830,879; 6,258,787; 6,121,246; RE37,933, 5,851,521 and 5,106,386; the disclosures of which are incorporated herein by reference.

In one approach, and to simplify the manipulation and handling of the nucleic acid encoding the ezrin modulating agent, the nucleic acid is preferably inserted into a cassette where it is operably linked to a promoter. The promoter must be capable of driving expression of the mitogen in the desired target host cell. The selection of appropriate promoters can readily be accomplished. Preferably, one would use a high expression promoter. An example of a suitable promoter is the 763-base-pair cytomegalovirus (CMV) promoter. The Rous sarcoma virus (RSV) (Davis, et al., Hum Gene Ther 4:151 (1993)) and MMT promoters may also be used. Certain proteins can expressed using their native promoter. Other elements that can enhance expression can also be included such as an enhancer or a system that results in high levels of expression such as a tat gene and tar element. This cassette can then be inserted into a vector, e.g., a plasmid vector such as pUC 118, pBR322, or other known plasmid vectors, that includes, for example, an E. coli origin of replication. See, Sambrook, et al., Molecular Cloning: A Laboratory Manual, Cold Spring Harbor Laboratory press, (1989). The plasmid vector may also include a selectable marker such as the beta.-lactamase gene for ampicillin resistance, provided that the marker polypeptide does not adversely effect the metabolism of the organism being treated. The cassette can also be bound to a nucleic acid binding moiety in a synthetic delivery system, such as the system disclosed in WO 95/122618.

If desired, the DNA may also be used with a microdelivery vehicle such as cationic liposomes and adenoviral vectors. For a review of the procedures for liposome preparation, targeting and delivery of contents, see Mannino and Gould-Fogerite, Bio Techniques, 6:682 (1988). See also, Feigner and Holm, Bethesda Res. Lab. Focus, 11 (2):21 (1989) and Maurer, R. A., Bethesda Res. Lab. Focus, II (2):25 (1989).

Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

Thus in one embodiment, the present invention method further includes administering at least one nucleic acid described herein with a stent, catheter, implementation for performing balloon angioplasty; or related device known to those working in the field. In one embodiment, the methods are employed to reduce or eliminate ischemia in myocardial or related tissue that is known, suspected to be, or at risk of being impacted by one or more of ischemia, infarction or dysfunction. More generally, vascular ischemia such as those ailments impacting limbs can be treated or prevented by use of the invention.

Methods for detecting an increase or a decrease in ezrin DNA binding activity can be monitored by one or a combination of approaches including the specific assays provided herein. In one approach, the decrease in ezrin DNA binding activity is at least about 50%, preferably about 70%, more preferably at least about 90% up to about 100% as determined by what is referred to herein as a “standard cyclin A promoter binding assay”. That assay monitors the amount of nuclear ezrin available for promoter binding in an in vitro test. Typically, the assay will involve at least one an preferably all of the following steps:

1) isolating nuclear proteins from subject cells,

2) separating the proteins electrophoretically and transferring the separated proteins to a suitable solid support,

3) probing the solid support with an oligonucleotide (preferably detectably-labeled) that is substantially identical to at least part of a mammalian cyclin A promoter (eg., the DNA binding part of the CHR element); wherein the probing is conducted under conditions suitable for forming a specific binding pair between the oligonucleotide and any ezrin in the isolated nuclear proteins; and

4) detecting, and optionally quantifying, presence of the binding pair as being indicative of the DNA binding activity of the ezrin.

A preferred assay is conducted in what has been referred to as a “Southwestern” format. See the Examples section below. If desired, the assay is readily adapted to test tissue samples. Detection can be accomplished by a variety of standard methods including densitometric analysis.

Suitable control experiments for use with assays describe herein include replacing the cyclin A gene or fragment with an unrelated nucleic acid sequence including, but not limited to, nucleic acid sequence obtained from a bacterial or viral vector eg., pBR322 or a fragment thereof.

The Southwestern assay just described is flexible and can be used to confirm ezrin binding to the mammalian cyclin A promoter. Alternatively, or in addition, the assay can be used to test one or a combination of ezrin modulating agents (eg., fragments or derivatives of TNFsr, TNF blocker (antagonist), and anti-TNF antibody) for capacity to decrease or totally block the DNA binding activity of ezrin. Preferred fragments and derivatives of TNFsr, TNF blocker (antagonist) and the anti-TNF antibody are capable of reducing ezrin DNA binding activity by at least about 80% compared to the corresponding full-length or non-derivatized TNFsr, TNF blocker (antagonist) or anti-TNF antibody sequence.

In another assay format, one or a combination of suitable oligonucleotides including at least part of the mammalian cyclin A gene (eg., the DNA binding part of the CHR element) is coupled to a solid support. An example of a such a support is a “chip” suitable for use in surface plasmon resonance or a related approach. In one invention embodiment, mammalian ezrin or an effective fragment or derivative thereof is coated to a suitable biosensor chip (Pharmacia) as directed. Interaction between the ezrin or fragment thereof and the oligonucleotide can be registered by the biosensor. Such as assay can be used to confirm ezrin binding and/or can be employed to screen for ezrin modulating agents. This invention embodiment finds particular use in situations where “high throughput” screening strategies are needed to detect agents such as those present in “libraries” of such agents.

Additionally suitable ezrin modulating agents according to the invention increase or decrease phosphorylation of mammalian ezrin, preferably by phosphorylating at least one tyrosine, threonine or serine residue therein. Method for detecting and optionally quantifiying protein phosphorylation are known in the field and include routine immunological assays. Such assays include but are not limited to tests in which a first antibody is used to bind phosphotyrosine or phosphoserine specifically followed by use of a second antibody to bind the first antibody. Presence (or absence) of phosphorylation is detected with respect to a suitable control. Such antibodies can be used alone or in combination with a suitable label such as biotin. Reference herein to a “standard protein phosphorylation assay” generally refers to this “sandwich” type immunoassay. See Ausubel et al., Current Protocols in Molecular Biology, John Wiley & Sons, New York (1989); Harlow and Lane in, Antibodies: A Laboratory Manual (1988); and the Sigma-Aldrich catalogue (St. Louis, Mo. 2003) for disclosure relating to these methods.

Substantial identity between two nucleic acid molecules can be readily ascertained by using conventional computer software programs such as BLAST, FASTA and the like.

Still another approach referred to herein as a “standard ezrin mRNA stability assay” or like phrase can be used to monitor ezrin activity by detecting and optionally measuring the stability of mRNA encoding mammalian ezrin. In a specific approach, stability of the ezrin mRNA is monitored by isolating RNA from a desired cell (or tissue) and probing same with a cyclin DNA template. Subsequently, a standard ribonuclease protection assay (RPA) is conducted. RNAse protected transcripts are then precipitated, separated electrophoretically, and then detected and optionally quantified by conventional methods. Preferably the decrease in ezrin activity detected by the standard ezrin mRNA stability assay is at least about 20%, preferably at least about 50%, more preferably about 80%, and still more preferably up to about 100% as determined by the assay. A preferred example of such an assay is described below in the Examples section.

In embodiments in which the invention is used to decrease the proliferation of endothelial cells and progenitor cells, preferably the decrease is at least about 20% as determined by a standard restenosis assay, preferably at least about 50%, more preferably at least about 80% as determined by that assay. Restenosis is recognized as a post-surgical complication particularly of angioplasty and related vascular intervention therapies. Methods for detecting and optionally quantifying restenosis are known. See eg., Landau., C et al. (1994) N. Eng. J. Med. 330:981; Tardif J. C et al. (1997) N. Eng. J Med. 337:365: Ferns, G. A. et al. (1992) PNAS (USA) 89:11312; and references cited therein. Preferred methods are described in Krasinski, K. et al. (2001), supra.

A particular mammal in accord with this invention is a primate, rodent, or domesticated animal such as a rabbit, horse, cow, pig, dog, cat, goat, sheep and the like. A preferred primate is a human patient eg., such as one that is suspected of having, or will have ischemic tissue. Typical ischemic tissue is associated with an ischemic vascular disease as described herein.

As discussed, it will sometimes be useful to contact ECs and/or EPCs with one or a combination of ezrin modulating agents under conditions sufficient to decrease ezrin activity therein followed by administering the cells to a mammal in need of such treatment. Such an “ex vivo” approach may be particularly indicated in treatment of human patients (eg., treating or preventing ischemia or related condition) where robust growth of new or existing blood vessels is needed. In embodiments in which the agent is a nucleic acid, the contact involves transforming the ECs and/or EPCs (transiently or long-term) with the nucleic acid under conditions conducive to expressing the agent in the cells. Typically, such cells can be administered to the mammal in need of such treatment. General methods for isolating EC and EPC cells including ex vivo manipulations have been disclosed. See eg., PCT application WO 99/45775 to J. Isner and references cited therein. See also U.S. Pat. Nos. 5,980,887; 6,258,787; and 6,121,246. By “long term” is meant at least about a few days, preferably at least about a week or longer up to about a few months.

Preferred ECs and EPCs for use with the present invention are characterized by having at least one of and preferably all of the following markers: CD34⁺, flk-1⁺, and tie-2⁺. See eg., U.S. Pat. Nos. 6,659,428; 5,980,887; EP1061800; WO 99/45775; 5,830,879; 6,258,787; 6,121,246; RE37,933, 5,851,521 and 5,106,386, (disclosing methods for preparing and using ECs and EPCs eg., to promote blood vessel growth).

In one approach, ECs are isolated from a human patient and transformed with nucleic acid encoding one or more dominantly and negatively acting fragments of the human ezrin protein eg., amino acid positions 1 to about 150, preferably between about 1 to about 115 as described previously; or a derivative thereof. In this invention embodiment, ezrin activity in the transformed ECs will decrease, thereby stimulating cyclin A synthesis and cell proliferation.

In embodiments in which it is desired to enhance angiogenesis in a mammal, the present methods can be used alone or in combination with at least one suitable cytokine, angiogenic or hematopoietic protein or effective fragment as disclosed in the PCT application WO 99/45775 by Isner, J. et al. and references cited therein. See also the U.S. Pat. Nos. 5,980,887; 6,258,787; and 6,121,246 for additional and related disclosure.

By the phrase angiogenic protein is meant acidic fibroblast growth factor (aFGF), basic fibroblast growth factor (bFGF), vascular endothelial growth factor (VEGF-1), epidermal growth factor (EGF), transforming growth factor α and β (TGF-α and TFG-β), platelet-derived endothelial growth factor (PD-ECGF), platelet-derived growth factor (PDGF), tumor necrosis factor α (TNF-α), hepatocyte growth factor (HGF), insulin like growth factor (IGF), erythropoietin, colony stimulating factor (CSF), macrophage-CSF (M-CSF), angiopoetin-1 (Ang1) or nitric oxidesynthase (NOS); or an effective fragment thereof.

A preferred angiogenic protein for use with the invention is VEGF-B, VEGF-C, VEGF-2, VEGF-3; or an effective fragment thereof.

Alternatively, or in addition, the methods described herein can be used in combination with at least one hematopoietic protein. Reference herein to a hematopoietic protein means granulocyte-macrophage colony-stimulating factor (GM-CSF), VEGF, Steel factor (SLF, also known as Stem cell factor (SCF) ), stromal cell-derived factor (SDF-1), granulocyte-colony stimulating factor (G-CSF), HGF, Angiopoietin-1, Angiopoietin-2, M-CSF, b-FGF, and FLT-3 ligand; or an effective fragment thereof.

Methods for testing and identifying a variety of suitable cytokines, angiogenic or hematopoietic protein fragments have been reported in EP1061800 and WO99/45775, for instance.

As discussed, the invention is particularly useful for preventing, treating or reducing the severity of restenosis as well as related vasculopathies. Typical blood vessel damage is restenosis associated with an invasive manipulation eg., balloon angioplasty, or deployment of stent or catheter. An illustrative stent is an endovascular stent. In embodiments in which the invention is used to address restenosis, the present methods can be used alone or in combination with conventional therapies such as use of probucol. See generally Harrison's Principles of Internal Medicine (1991) 12 ed. McGraw-Hill, Inc.

As also mentioned the invention can be used to prevent or reduce the severity of blood vessel damage in a mammal such as a human patient. In one embodiment, the method includes decreasing ezrin activity in endothelial cells (EC) and/or EPCs, at least prior to (prior to, during or after) a time in which the mammal is exposed to conditions conducive to damaging the blood vessels in which the decrease in ezrin activity is sufficient to reduce the severity of the blood vessel damage in the mammal. Typical methods further include administering to the mammal at least one ezrin modulating agent sufficient to decrease ezrin DNA binding activity relative to a control.

In one invention embodiment, the ezrin modulating agent is injected at or near the site of blood vessel damage in the mammal. Examples of such agents have already been described and include one or more inhibitors of the rho kinase (eg.,Y27632). Additionally suitable agents include one or a combination of suitably dominant and negatively acting fragments of mammalian ezrin and particularly human ezrin; or a derivative thereof.

Blood vessel damage suitable for treatment or prevention according to the invention can be, for instance, restenosis associated with an invasive manipulation or associated with ischemia. The invasive manipulation can be directly or indirectly associated with balloon angioplasty, or deployment of stent or catheter such as an endovascular stent. The ischemia can be directly or indirectly associated with one or a combination of infection, trauma, graft rejection, cerebrovascular ischemia, renal ischemia, pulmonary ischemia, limb ischemia, ischemic cardiomyopathy, infarct or myocardial ischemia.

In embodiments in which one or a combination of ezrin modulating agents is to be administered to a mammal, the agent can be given at least about 12 hours before exposing the mammal to the conditions conducive to damaging the blood vessels such as about 1 to 10 days before exposing the mammal to the conditions conducive to damaging the blood vessels. If desired, the method can further include administering the ezrin modulating agent to the mammal following the exposure to the conditions conducive to damaging the blood vessels.

Administration of the ezrin modulating agent in accordance with the invention can be via injection, e.g., intraperitoneal or intravenous injection. In embodiments in which the agent is an amino acid sequence, such sequences are preferably produced synthetically or from mammalian cells or other suitable cells and purified prior to use to be essentially or completely free of pyrogens. The optimal dose for a given therapeutic application can be determined by conventional means and will generally vary depending on a number of factors including the route of administration, the patient's weight, general health, sex, and other such factors recognized by the art-skilled including the extent (or lack ) of cell proliferation and/or cycling desired to address a particular medical indication.

Administration can be in a single dose, or a series of doses separated by intervals of days or weeks. The term “single dose” as used herein can be a solitary dose, and can also be a sustained release dose. The subject can be a mammal (e.g,. a human or livestock such as cattle and pets such as dogs and cats) and include treatment as a pharmaceutical composition which comprises one or a combination of ezrin modulating agents. Such pharmaceutical compositions of the invention are prepared and used in accordance with procedures known in the art. For example, formulations containing a therapeutically effective amount of one ezrin modulating agent may be presented in unit-dose or multi-dose containers, e.g., sealed ampules and vials, and may be stored in a freeze dried (lyophilized) condition requiring only the addition of the sterile liquid carrier, e.g. water injections, immediately prior use.

For instance, administration of at least one ezrin modulating agent according to the invention can be in amounts ranging between about lpg/gram body weight to 100 mg/gram body weight. Precise routes and amounts of administration will vary according to intended use and parameters already discussed.

The invention can be used to decrease or eliminate vascularization in settings where an increase in blood supply is not wanted. Examples of such settings include, but are not limited to, cancer (benign and metastatic), vasculopathies such as atherosclerosis and related pathologies involving unwanted neovascularization. In particular, uncontrolled proliferation of vascular cells (EC and EPC) can substantially contribute to disease by occluding blood progress and increasing vessel remodeling.

Thus in one invention embodiment, the mammal subjected to one or more of the present methods is suspected of having, or is pre-disposed (eg, by family history or exposure to carcinogens) to develop cancer. The methods can be used alone or in combination with at least one chemotherapeutic drug or treatment intended to prevent, treat, or reduce the severity of that cancer in the mammal (eg., a nucleoside analog, mustard agent, cisplatin or radiation treatment).

Thus in a more general embodiment, the invention provides a method for decreasing angiogenesis in a mammal in which the method includes increasing ezrin activity in endothelial cells (ECs) of the mammal sufficient to decrease the angiogenesis. Typical methods further include administering to the mammal at least one ezrin modulating agent sufficient to decrease ezrin DNA binding activity relative to a control. As an illustration, the ezrin modulating agent can be injected at or near a site for which the descrease in angiogenesis is desired. A preferred ezrin modulation agent is TNF Necrosis Factor alpha (TNF-α), rho kinase; or an effective fragment or derivative thereof.

As also mentioned, the invention provides a method for detecting a compound that modulates ezrin activity that includes in one embodiment introducing nucleic acid encoding at least part of the human cyclin A gene linked to a sequence encoding a detectable label; adding at least one known or candidate ezrin modulating compound to the cells; culturing the cells under conditions conducive to expressing the nucleic acid; measuring the detectable label in the presence and absence of the compound; and determining the effect of the compound on the cells.

The foregoing screening method is flexible and is compatible with use of one or a combination of nucleic acids encoding the human cyclin A gene. A particular nucleic acid of interest includes a region spanning about −1200 to about +250 of the human cyclin A gene, preferably about −900 to +150 of the gene, more preferably about −200 to about +100 of that gene. Even more preferably the nucleic acid includes at least one of the AP1, ATF and CDE-CHR binding sites as discussed in the Drawings and the Examples section, more preferably at least the CHR element. A preferred gene is human cyclin A although other mammalian gene sequences may be just as suitable for some applications.

The foregoing method is compatible with use of a wide spectrum of detectable labels. Such labels include, but are not limited to, those sequences known to encode a detectable protein sequence such as green fluorescent protein (GFP) or red fluorescent protein (RFP) from certain well known jellyfish. Other acceptable sequence labels include luciferase and the beta-galactosidase enzyme. Thus in embodiments in which the nucleic acid includes the cyclin A protein gene promoter spanning about −79 to +100, a preferred nucleic acid is covalently linked in-frame to a sequence encoding the luciferase or beta-galactosidase enzyme; or a functional fragment thereof.

By the phrase “ezrin DNA binding fragment” or related phrase is meant a part of the ezrin protein that binds the cyclin A gene, preferably the gene promoter portion, and specifically at least the CHR fragment of the cyclin A gene promoter. See the Examples that follow. Methods for detecting such binding are standard and involve Southwestern type assays. In one approach, such fragments are readily identified by conducting at least one and preferably all of the following steps:

a) contacting, to the cyclin A gene, preferably the CHR fragment thereof, the subject ezrin protein fragment, the contacting being under conditions sufficient to form a specific binding pair,

b) forming a specific binding pair between the cyclin A gene (or fragment) and the subject fragment of the ezrin protein; and

c) detecting the binding pair as being indicative of the presence of the ezrin DNA binding fragment. A preferred example of such a fragment will exhibit a binding affinity for the cyclin A gene (or fragment), measured eg., as a Kd, that is at least about 80%, preferably at least about 90% of that obtained with the substantially full-length ezrin protein (see Table I, for instance). Such an assay can be performed using surface plasmon resonance, for instance.

By the phrase “specific binding” or similar term is meant a molecule disclosed herein which binds another molecule, thereby forming a specific binding pair, but which does not recognize and bind to other molecules as determined by, e.g., Western blotting, ELISA, RIA, gel mobility shift assay, enzyme immunoassay, competitive assays, saturation assays or other suitable protein binding assays known in the art. See generally Ausubel et al. supra, Sambrook et al. supra, and Harlow and Lane Antibodies: A Laboratory Manual, CSH Publications, N.Y. (1988), for suitable conventional methods for detecting specific binding in a variety of formats.

By “derivative of the ezrin protein” or related phrase is meant an amino acid sequence with at least about 70% identity to the sequence shown in Table 1, preferably at least about 85% identify and more preferably at least about 95% up to about 99% identity. Identity between two amino acid sequences is readily determined by inspection or more preferably by use of conventional computer programs such as BLAST.

As already mentioned, the invention provides a method for detecting DNA binding between ezrin (or a DNA binding fragment thereof) and at least part of a mammalian cyclin A gene. In one embodiment, the method includes at least one and preferably all of the following steps:

1) incubating at least part of a mammalian cyclin A gene, with the ezrin protein or a DNA binding fragment thereof, wherein the incubation is conducted under conditions sufficient to form a specific binding pair between the cyclin A gene and the ezrin protein (or fragment),

2) adding at least one known or candidate ezrin modulating compound to the incubation medium; and

3) detecting presence of a specific binding pair between the cyclin A gene (or fragment) and the ezrin protein (or fragment) in the presence and absence of the compound, wherein a reduction or absence of the binding pair is taken to be indicative of a compound that reduces or blocks ezrin binding to the cyclin A gene.

In a particular embodiment, the part of the part of the cyclin A gene is a detectably-labeled oligonucleotide comprising at least the CDE-CDR sequence. The detectable label can be visualized by nearly any acceptable means including an automated or semi-automated fluorescence, colorimetric, or phosphorescence detection device.

As shown above and in the following examples, the invention addresses a new basis of TNF-mediated suppression of cyclin A particuarly in EC cells. The Examples show that TNF specifically down-regulates cyclin A mRNA and that this down-regulation is mediated both by a decreased transcription as well as via enhanced degradation of cyclin A mRNA. Without wishing to be bound to theory, the examples are also believed to show that CDE-CHR co-repressor cis elements in the cyclin A promoter are the targets of TNF-mediated transcriptional repression. The examples also show that TNF induces a functionally novel 84-kDa ezrin protein that binds specifically to CHR co-repressor element in the cyclin A promoter.

In general, preparation of the fusion molecules of the invention includes conventional recombinant steps involving, e.g., polymerase chain amplification reactions (PCR), preparation of plasmid DNA, cleavage of DNA with restriction enzymes, preparation of oligonucleotides, ligation of DNA, isolation of mRNA, introduction of the DNA into a suitable cell, and culturing of the cell. Additionally, the fusion molecules can be isolated and purified using chaotropic agents and well known electrophoretic, centrifugation and chromatographic methods. See generally, Sambrook et al., Molecular Cloning: A Laboratory Manual (2nd ed. (1989); and Ausubel et al., Current Protocols in Molecular Biology, John Wiley & Sons, New York (1989) for disclosure relating to these methods.

As discussed, the invention is well-suited for detecting candidate compounds that modulate (increase or preferably decrease) ezrin activity. In one embodiment, the nucleic acid introduced in the cells (which can be EC, EPC or other suitable cells such as HeLa, CHO, ect) encodes the cyclin A protein (or fragment thereof) covalently linked in-frame to a fluorescent or phosphorescent protein label. Examples of such acceptable labels are well-known in the field and include those derived from a fluorescent jellyfish protein, preferably green fluorescent protein (GFP) or red fluorescent protein (RFP). Such proteins and constructs encoding same are commercially available. Additionally acceptable nucleic acids encode the cyclin A protein or fragment covalently linked in-frame to a suitable enzyme detection system such as those involving luciferase, or beta-galactosidase enzyme.

In embodiments in which southwestern type detection formats are preferred the part of the cyclin A gene is typically a detectably-labeled CHR oligonucleotide, preferably radioactively labeled See the Examples below.

As mentioned, the invention further provides a pharmaceutical product for inducing neovascularization in a mammal. In one embodiment, the product comprises endothelial cells and/or EPCs, and the product includes at least one ezrin modulating agent. Preferably, the product and especially the cells are formulated to be physiologically acceptable to a mammal. Also preferably, the product is sterile and optionally includes at least one angiogenic or hematopoietic protein or nucleic acid encoding the protein. Alternatively, or in addition, the endothelial cells have been manipulated to express one or more ezrin modulating agents (transiently or long term).

Further provided by the invention are kits adapted for introducing endothelial cells and/or EPCs into a mammal. In one embodiment, the kit includes at least one ezrin modulating agent and optionally at least one angiogenic or hematopoietic protein or nucleic acid encoding same. The kit may further include a pharmacologically acceptable carrier solution, nucleic acid or mitogen, means for delivering the cells and directions for using the kit. Acceptable means for delivering the cells include transfer by stent, catheter or syringe.

Protein and nucleic acid sequences not specifically disclosed herein can be found in GenBank. See the National Center for Biotechnology Information (NCBI) at the National Library of Medicine, 38A, 8N05 Rockville Place, Rockville, Md. (USA) 20894. Alternatively, or in addition, such sequence information can be obtain from EMBL and/or SWISS-PROT. Using this information, a DNA or RNA segment encoding the desired sequence may be chemically synthesized or alternatively, in which the sequence is DNA or RNA, the sequence may be obtained using routine procedures such as PCR amplification.

The invention is further illustrated by reference to the following non-limiting examples.

### EXAMPLE 1

EC Cyclin A mRNA is Specifically Down-Regulated by TNF

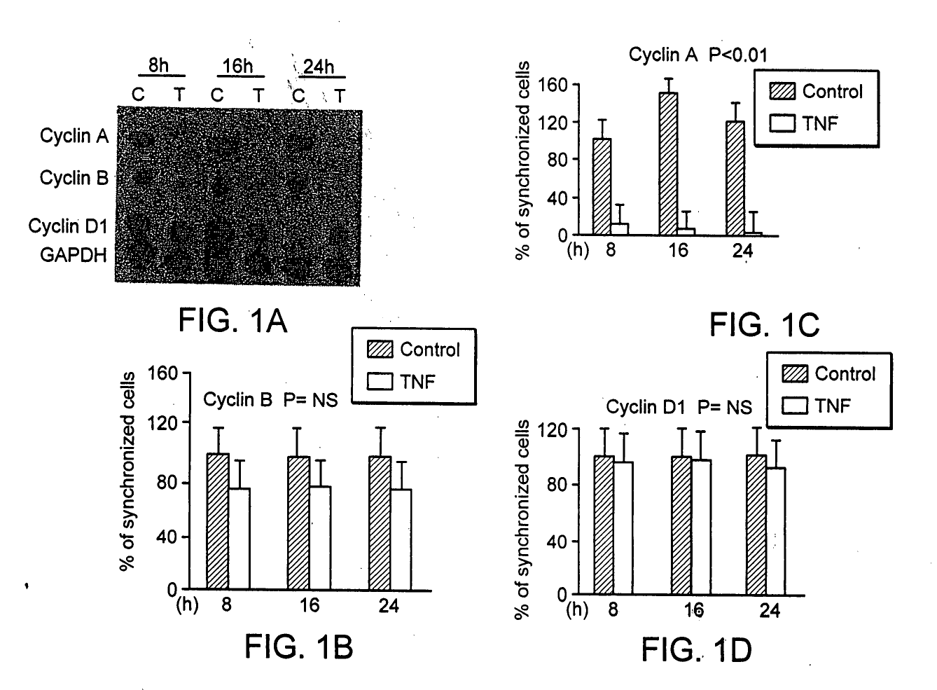

It has been shown that treatment of EC with TNF suppressed cyclin A protein levels as well as cyclin dependent kinase (cdk) 2 activity¹⁵. To determine the specificity of this suppression MRNA expression of cyclins A, B and D1 in EC exposed to TNF was analyzed. As shown in FIGS. 1A-D, cyclin A mRNA was almost undetectable after 8 h TNF treatment. However, TNF exposure resulted in no discernible changes in the mRNA levels of cyclin B and cyclin D1. These data show that TNF specifically down-regulates cyclin A mRNA expression in EC.

FIGS. 1A-D are explained in more detail as follows. Synchronized ECs were serum stimulated in the presence or absence of TNF for indicated times. Total cellular RNA was isolated and was analyzed by RPA for cyclin A, cyclin B and cyclin D1 expression. A representative autoradiograph is shown (FIG. 1A). mRNA expression of cyclins was corrected to that of GAPDH and quantified by densitometric analysis (FIGS. 1B-D). Data represent average of 3 similar experiments.

### EXAMPLE 2

TNF Suppresses De Novo mRNA Synthesis of Cyclin A

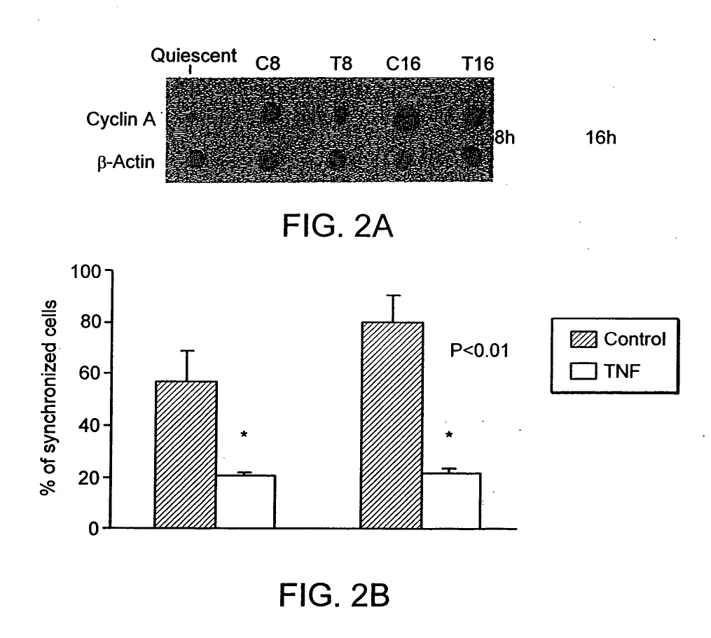

To investigate whether TNF induced repression of cyclin A mRNA reflects a decreased transcriptional rate, nuclear run-on experiments were carried out. Serum stimulation of synchronized BAEC stimulated de novo cyclin A mRNA synthesis (FIGS. 2A-B). However, exposure of cells to TNF for 8-16 h blunted the de novo cyclin A mRNA transcription 5 fold at 16 h. There was no effect of TNF on control β-actin mRNA synthesis.

FIGS. 2A-B are explained in more detail as follows. In FIG. 2A, nuclei harvested from quiescent and serum stimulated EC cultured with or without TNF were subjected to nuclear run-on analysis. Radiolabeled RNA was hybridized with cyclin A cDNA immobilized on nylon membranes and de novo transcribed cyclin A was detected by autoradiography. Data from 3 similar experiments was quantified by densitometric analysis in FIG. 2B.

### EXAMPLE 3

Cyclin A mRNA is Specifically Destabilized by TNF

Although TNF reduces the cyclin A mRNA transcription, decrease in the rate of de novo mRNA synthesis at 16 h post TNF treatment (see Example 2) does not account for the total loss of cyclin A mRNA at the same time points as observed earlier (see Example 1). These data indicated the presence of additional TNF-mediated suppressive mechanism(s).

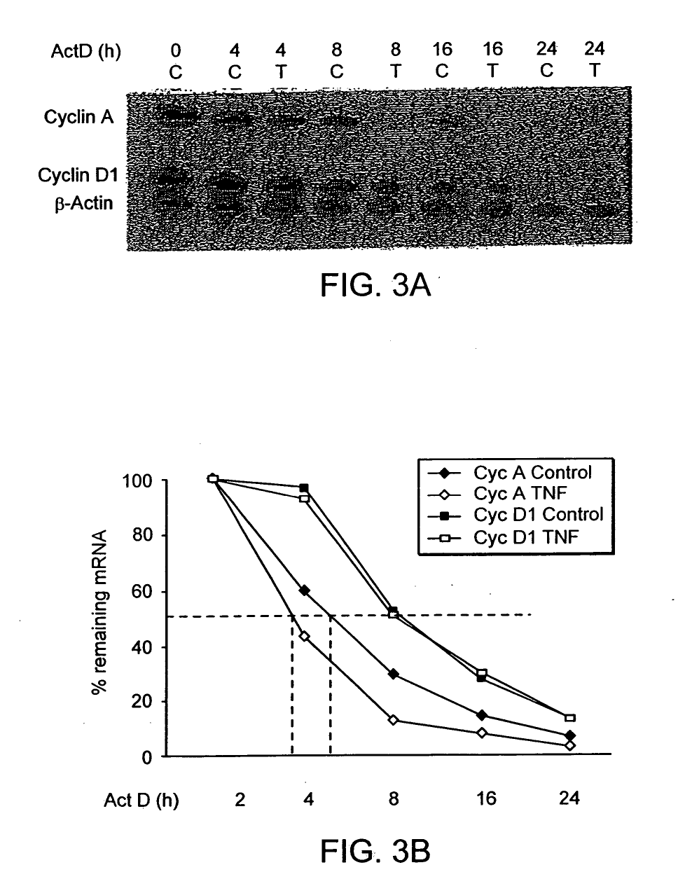

Since TNF is known to influence mRNA stability and since cyclin A regulation also occurs at the level of mRNA stability, the influence of TNF on cyclin A mRNA stability was investigated Cells were stimulated with serum, with or without TNF and in the presence of actinomycin D (5 μg/mL) for 4-24 h. RNA was isolated and analyzed by RPA for cyclin A, cyclin D1 and GAPDH mRNA levels. As shown in FIGS. 3A-B, under basal condition cyclin A mRNA decayed with an approximate half-life of 5.5 h. However, the half-life of cyclin A mRNA was reduced to approximately 3.0 h in the presence of TNF. There was no significant change in the half-life of cylin D1 mRNA in response to TNF, which decayed, with an approximate half-life of 8 h in the presence or absence of TNF. These results show that both transcriptional and post-transcriptional mechanisms mediate TNF suppressive action on cyclin A gene expression.

FIGS. 3A-B are explained in more detail as follows. FIG. 3A is a representation of an autoradiograph in which total RNA (5-10 μg) from ECs treated or not with TNF in the presence or absence of actinomycin D was hybridized with in vitro transcribed antisense cyclin multiribo-probe and analyzed by RPA. A representative autoradiograph of 3 similar experiments is shown. FIG. 3A is a graph representing quantified values for % remaining cyclin A and cyclin D1 mRNA at indicated time points, normalized with that of β-actin control.

### EXAMPLE 4

CDE-CHR Cis-Elements are Essentialfor Efficient Cyclin A Promoter Activity

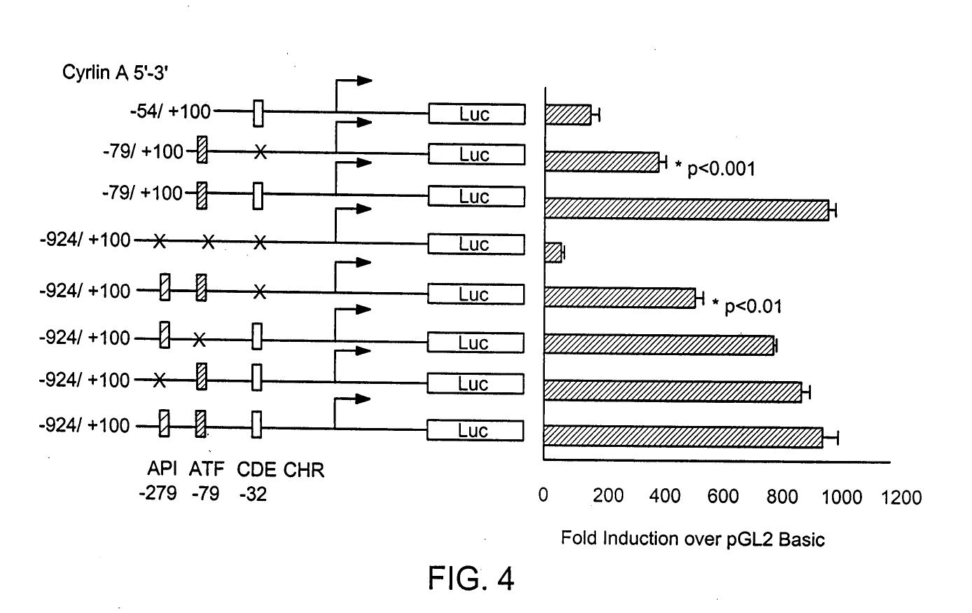

The present example addresses TNF mediated transcriptional suppression of cyclin A. The requirement of individual transcription factor binding sites for the transcription of cyclin A was investigated using reporter constructs containing various fragments of the cyclin A promoter (FIG. 4) and its binding site deletion mutants, linked to a luciferase reporter gene. EC were transiently transfected with each of the reporter constructs and fold induction of reporter activity over control pGL2 vector was documented.

As indicated in FIG. 4, a reporter construct encompassing −924 to +100 region of cyclin A promoter and a shorter fragment containing regions −79 to+100 were equipotent in driving the transcription of the reporter gene. A fuirther truncation of promoter (−54/+100) rendered the promoter ineffective, suggesting minimal requirement of at least −79/+100 region for optimal promoter activity. These data show that cis-elements within −79/+100 region of cyclin A promoter harbor elements that are both necessary and sufficient for effective cyclin A transcription. This conclusion was supported by the observation that deletion of CDE elements from both −924/+100 and −79/+100 fragments repressed cyclin A promoter activity (p<0.01 and 0.001, respectively). Furthermore, deletion of either AP1 or ATF binding sites from the −924/+100 fragment did not significantly suppress promoter activity. As expected removal of all three binding sites (AP1, ATF and CDE-CHR) from −924/+100 promoter completely abolished the promoter activity. These data confirm the finding of other studies^{12, 16, 17} that CDE-CHR cis-elements in the cyclin A promoter are essential for efficient transcription of cyclin A.

FIG. 4 is explained in more detail as follows. EC were transiently transfected with luciferase reporter promoter constructs containing 5′ cyclin A sequences as shown. Following 24 h post-transfection, media was collected and analyzed for luciferase activity. Data represent the luciferase activity observed in five independent transfections (mean±SEM), normalized to alkaline phosphatase activity produced by a co-transfected control plasmid (pSVAPAP).

### EXAMPLE 5

TNF-Mediated Suppression of Cyclin A Promoter Activity Requires CDE-CHR Elements

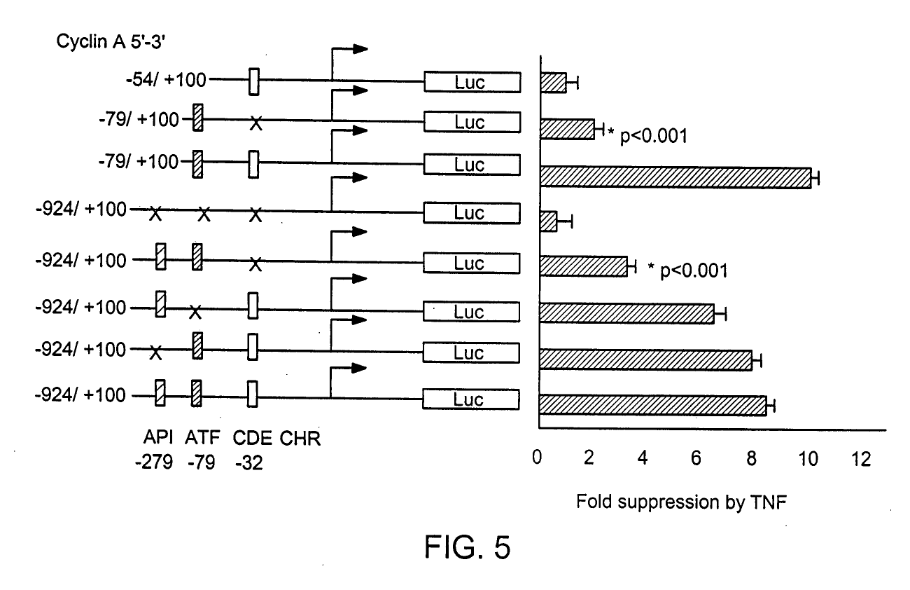

To delineate the cis-elements that may be the target of TNF suppressive action, a subset of BAEC transiently transfected with reporter constructs as shown in FIG. 4, were treated with TNF, prior to the analysis of reporter activity. As shown in FIG. 5, reporter activity was significantly suppressed by TNF in cells transfected with those constructs that contained CDE-CHR elements. Deletion of either AP1 or ATF binding sites from −924/+100 construct had no effect on TNF-mediated repression of promoter activity. However, deletion of CDE-CHR significantly abrogated the suppressive action of TNF on cyclin A promoter activity (p<0.001). The results show that CDE-CHR elements are not only required for optimal promoter activity but are also the target of TNF-mediated suppression of cyclin A promoter activity.

FIG. 5 is explained as follows. Subsets of ECs transfected with reporter constructs were cultured further in the presence of TNF prior to the analysis of reporter activity. Bars represent TNF-mediated average fold suppression of promoter activity in transfectants from previous experiment shown in FIG. 4. Data represents average of 5 similar experiments (mean±SEM).

### EXAMPLE 6

TNF Modulates CDE-CHR Binding Activity

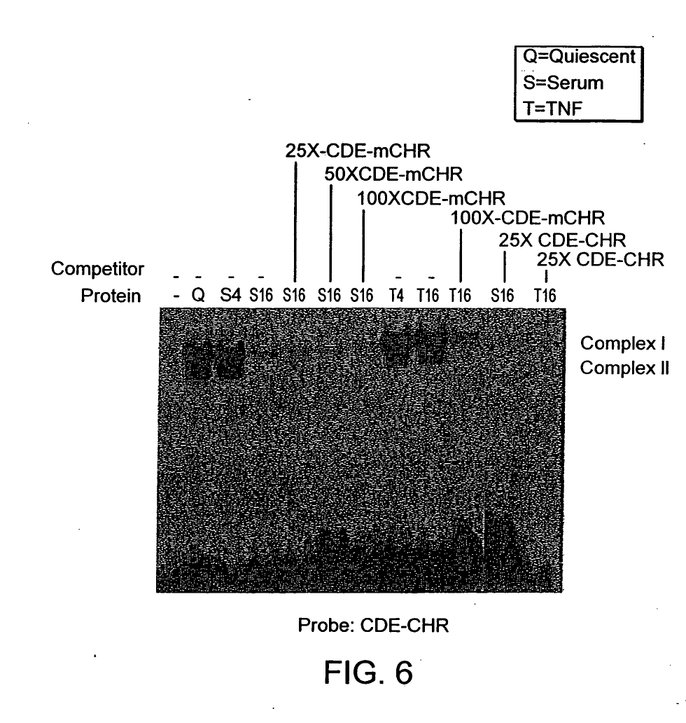

The present example addresses whether TNF could modulate the binding of transcription factors to the CDE-CHR elements in functional assays. Specifically, EMSAs were carried out using synthetic oligonucleotides spanning the CDE-CHR elements of the cyclin A promoter or their mutant forms wherein either CDE or the CHR sites were mutated. Nuclear extracts from synchronized EC formed two complexes with CDE-CHR oligonucleotides (FIG. 6). Serum stimulation of cells for 16 h completely abrogated complex II and greatly reduced complex I. These data show that in growth arrested cells, CDE-CHR repressor elements are occupied with trans-factors such as CDF-l thereby inhibiting transcription of cyclin A. Upon growth stimulation these factors are released, allowing the transcription to proceed^{16, 18}. In EC, TNF increased the binding activity of complex I both at 4 h and more appreciably at 16 h. Unlike that by serum stimulated extracts, complex I formation by TNF treated extracts, could not be competed by 25-50 fold and modestly competed by 100 fold excess of cold CDE-mCHR oligonucleotide. This indicates that complex I represents binding activity specific to CHR elements and not to both CDE and CHR elements like CDF-1. As a control 25 fold excess of self competition completely abrogated both complexes. These data support two models: either a sustained binding of factors to CHR, thereby repressing transcription of cyclin A despite serum stimulation, or recruitment/induction of additional protein/factor in response to TNF which keeps the CHR occupied and thereby represses promoter activity.

FIG. 6 is explained in more detail as follows. Nclear extracts from ECs treated or not with TNF were incubated with end-labeled oligonucleotide spanning CDE-CHR elements from cyclin A promoter. The binding reactions were then resolved on a 5% native PAGE in an EMSA assay. For competition experiments, 25-100 fold excess of indicated unlabeled oligonucleotide was incubated with nuclear extracts prior to the addition of radiolabeled probe. Representative autoradiograph of 3 similar experiments is shown.

### EXAMPLE 7

CHR and not the CDE Binding Activity is Modulated by TNF

To gain insight into the individual contribution of the CDE and/or the CHR elements, bindings assays were performed utilizing an oligonucleotide in which CDE site was nucleotide substituted and by competing the binding with oligonucleotide, with substitution mutations of either CDE (self) or that of CHR sites.

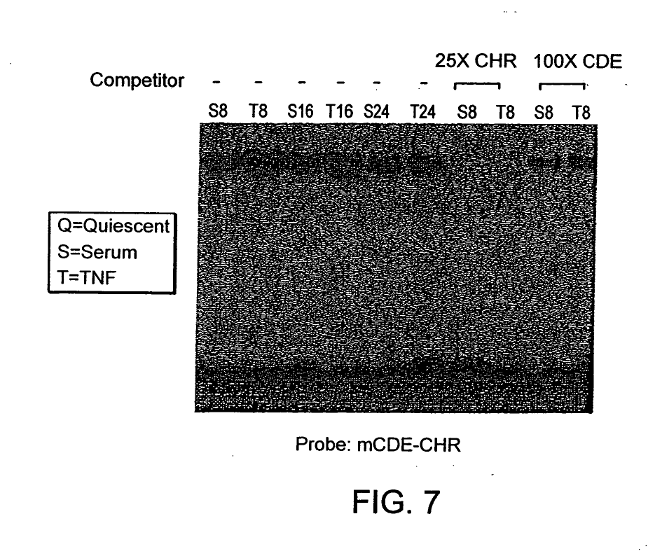

As depicted in FIG. 7, nuclear proteins from asynchronously growing BAEC formed a single complex with mobility similar to that of complex I in FIG. 6. This binding activity was increased in nuclear extracts from cells treated with TNF for 8 h. Furthermore; a 25 fold excess of unlabeled mCDE-CHR oligonucleotide effectively inhibited this binding activity whereas a 100 fold excess of unlabeled CDE-mCHR oligonucleotide did not. These data indicate that modulation of binding activity by TNF represents the binding of nuclear proteins to the CHR elements rather than to the CDE elements. EMSA using labeled CDE-mCHR probe also formed a single complex with no significant changes by TNF in the binding activity.

FIG. 7 is explained in more detail as follows. EMSAs were carried out using a variant of wild type CDE-CHR oligonucleotide ( CDE elements mutated) as the probe. Nuclear extracts from cells cultured in the presence or absence of TNF for indicated time points were used in the binding reaction. For competition experiments, 25 or 100 fold excess of unlabeled oligonucleotide was incubated with nuclear extracts prior to the addition of radiolabeled probe. A representative autoradiograph of 3 similar experiments is shown.

### EXAMPLE 8

TNF-induced 84 kDa Protein Binds Specifically to Cyclin A CHR Elements

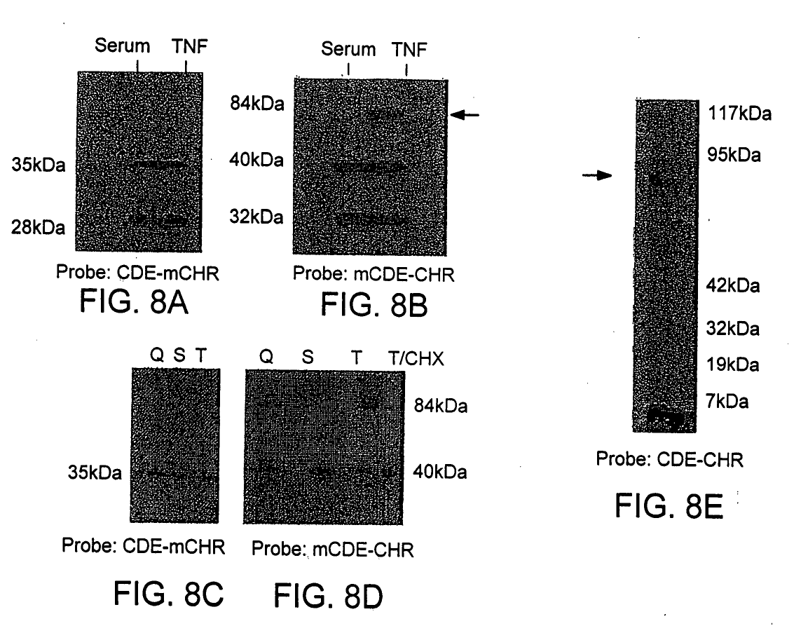

To identify the proteins, which formed complexes with CDE and CHR elements in EMSA, south-western experiments were carried out. Nuclear proteins from asynchronously growing BAEC with or without TNF were probed with ³²P-labeled CDE-mCHR or mCDE-CHR oligonucleotides. As shown in FIG. 8A, two proteins of approximately 35 and 27 kDa were detected with CDE-mCHR probe. However, levels of neither protein changed in response to TNF. Similarly, two proteins of approximately 39 and 30 kDa were observed in extracts from control cells, when probed with mCDE-CHR oligonucleotide. Interestingly, an additional protein of approximately 84 kDa was found to react with mCDE-CHR probe in the nuclear extracts from TNF treated cells. These results further confirm that modulation in the CHR binding activity by TNF was specific and due to the induction or modulation of a new protein.

### EXAMPLE 9

CHR Binding Complex Contains TNF Induced 84 kDa Protein

To confirm that 84 kDa protein in TNF treated extracts identified in FIGS. 8A-B, does bind to CHR elements, we performed UV cross-linking experiments. EMSA reactions were assembled as before followed by UV cross-linking. The reactions were then boiled in SDS loading buffer and resolved on 12% SDS-PAGE. In reactions probed with CDE-mCHR the 35-kDa protein seen before was observed again (FIGS. 8C-D). In reactions probed with mCDE-CHR probe, the previously identified (FIGS. 8A-B) 39-kDa protein was found cross-linked to oligonucleotide. Similarly, the 84-kDa protein from TNF treated extract cross-linked with mCDE-CHR probe, further confirming the southwestern results. Moreover, the induction of 84-kDa protein by TNF was protein synthesis dependent since treatment of cells with cycloheximide (5 μg/mL) along with TNF failed to induce the protein.

To further confirm that this TNF-induced 84 kDa protein is a constituent of CHR binding activity in TNF treated extracts, the specific band from mCDE-CHR EMSA was excised, UV cross-linked and eluted DNA-protein mixture was subjected to SDS-PAGE. The results, shown in FIG. 8E, confirm that TNF induced 84 kDa protein is indeed a constituent of CHR binding activity.

FIGS. 8A-E are explained in more detail as follows. FIGS. 8A-B. Representative autoradiograph of south-western analysis. Nuclear proteins from asynchronously growing EC with or with out TNF treatment were size fractionated on a 12% SDS-PAGE gel, transferred to PVDF membrane and renatured. Membranes were then incubated with radio-labeled CDE-mCHR or mCDE-CHR oligonucleotide. Membranes were washed, air dried and autoradiographed. FIGS. 8C-D. DNA Binding reactions using radio-labeled CDE-mCHR and mCDE-CHR oligonucleotides and nuclear proteins from quiescent and serum stimulated EC (±TNF) were exposed to short wavelength UV light (254 nm) for 20 min. UV cross-linked DNA-protein complexes were resolved on 12% SDS-PAGE gels, dried and autoradiographed. FIG. 8E. CHR specific complex observed in EMSA using proteins from TNF treated EC was excised and UV cross-linked. Proteins were eluted from gel and resolved by SDS-PAGE. Gel was dried and autoradiographed. Each experiment was repeated at least 3 times.

The Examples provide evidence that TNF suppress de novo cyclin A mRNA synthesis (FIG. 2). It also shows that CDE-CHR elements in cyclin A promoter have been identified as the a target of TNF suppressive action. In an example above, enhanced destabilization of cyclin A mRNA in response to TNF (FIG. 3) has been shown. Without wishing to be bound to theory, TNF may modulate the affinity of cyclin A-AREs to the two functionally different ARE binding proteins.

The Examples also show that TNF specifically up-regulates DNA binding activity to CHR elements. Results coincide with the altered DNA binding activity. Disruption of CDE-CHR site renders insensitivity to TNF both in terms of promoter activity as well as to DNA binding activity.

Also shown is that binding to the CHR can occur in the presence of mutations within CDE element. The existence of a DNA-protein complex on the CHR seems to preclude binding to CDE. Without wishing to be bound to theory, this could either be due to the binding of mutually exclusive complex (FIG. 6), or to the existence of hetero-oligomers with decreased affinity for the dual element. Capacity to renature the cyclin A CHR binding activity observed in our study, after electrophoresis through denaturing SDS-PAGE helped identification of a 84-kDa protein. See FIGS. 8A-E.

The 84 kDa CHR binding protein described in the Examples is different from either CDF-1 or CHF. Unlike CDF-1, this protein does not bind to CDE elements. Information indicates that the CHR binding protein is not CHF. For instance, and as shown in FIG. 8C-D, this is a TNF induced protein and is influenced by de novo protein synthesis. Additionally, in contrast to CHF, binding of this protein to CHR is not dependent on cell cycle; as evident by sustained binding activity at 16 h in TNF treated extracts (FIGS. 6 & 7). Furthermore, absence of this protein in quiescent cells (FIG. 8C-D) is in contrast to CHF which is found in abundance in quiescent cells. Finally, protein described in this study differs from CHF in apparent molecular mass (80-84 kDa vs. 90-95 kea CHF).

The Examples further indicate that the 84kDa polypeptide helps establish TNF regulated repression of cyclin A transcription in EC. Targeted disruption of this protein can be a therapeutic strategy to rescue EC proliferation, in vivo.

Local expression of Tumor Necrosis Factor-alpha (TNF) at the sites of arterial injury following balloon angioplasty, suppresses endothelial cell (EC) proliferation and negatively affect re-endothelialization of the injured vessel. We have previously reported that in vitro exposure of EC to TNF induced EC growth arrest and apoptosis. These effects were mediated, at least in part, by down-regulation of cell cycle regulatory proteins. Here we report potential mechanism(s) for TNF mediated suppression of cyclin A in EC. TNF exposure to EC completely abrogated cyclin A mRNA expression via mechanisms involving both transcriptional and post-transcriptional modifications. TNF inhibited de novo cyclin A mRNA synthesis and suppressed cyclin A promoter activity. Utilizing deletion mutants of human cyclin A promoter, we have identified CDE-CHR (Cell cycle Dependent Elements-Cell cycle genes Homology Region) region of cyclin A promoter as a target for TNF suppressive action. Experiments to investigate CDE-CHR binding proteins/factors revealed a TNF-mediated increase in specific DNA binding activity to the CHR elements. This increase in binding activity by TNF was mediated via the induction of a functionally novel 84-kDa protein that binds specifically to CHR in south-western assays. UV cross-linking and SDS-PAGE analysis of proteins eluted from specific complex confirmed the presence of this 84 kDa protein. Moreover, induction of this protein by TNF was protein synthesis dependent. Additionally, exposure of EC to TNF markedly reduced cyclin A mRNA stability. Targeted disruption of this protein could potentially be a therapeutic strategy to rescue EC proliferation, in vivo.

### EXAMPLE 10

Ezrin Binds to Cyclin A-CHR Elements.

TNF treatment of EC down-regulates cyclin A transcription via recruitment of an 80 kDa protein, which binds specifically to CHR co-repressor elements in the cyclin A promoter. Partial purification and Matrix Assisted Laser Desorption Ionization (MALDI) analysis identified this protein as ezrin, a member of the cytoskeletal ERM proteins. This was an intriguing finding since ezrin is not known to bind to promoter cis-elements and regulate transcription of any known gene. We investigated the ability of ezrin to bind cyclin A-CHR elements as follows.

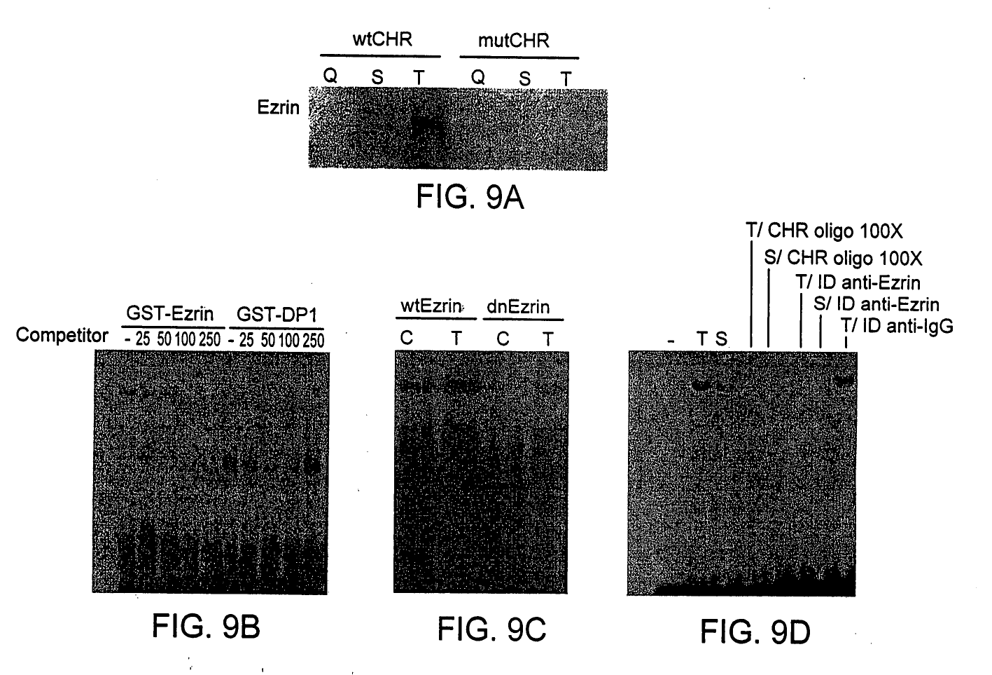

Nuclear extracts from control and TNF-treated EC were mixed with biotin-labeled CHR oligonucleotides and the bound proteins were captured on avidin-agarose beads. Eluted proteins from beads were subjected to Western blot analysis for ezrin. As shown in FIG. 9A, CHR oligonucleotides-nuclear protein complex revealed no detectable ezrin in quiescent cells, which was present at basal level in cells stimulated with serum but was substantially up-regulated in TNF-treated extracts. This CHR-ezrin interaction was specific since control oligonucleotides with mutations in the CHR region (mutantCHR) failed to capture ezrin from TNF-treated nuclear extracts. Specificity of CHR-ezrin binding was further confirmed by EMSA assays using recombinant-ezrin protein (GST-ezrin). Recombinant ezrin formed complex with CHR oligonucleotides, which was effectively competed by excess molar concentrations of the same unlabeled oligonucleotides (FIG. 9B). A control recombinant-DP1 protein did not form the complex of similar mobility with CHR oligonucleotides indicating the specificity of ezrin-CHR binding. We next evaluated the effect of TNF-induced ezrin binding to cyclin A-CHR by dominant-negative ezrin over-expression. EC were transiently transfected with wild-type (wtEzrin) and dominant negative (dnEzrin) ezrin expression vector. The efficacy of transgene expression was determined by quantifying expression of the VSVG protein tag in different transfectants and was found to be comparable between vectors and between different transfections (data not shown). Over-expression of dominant negative ezrin substantially abrogated TNF-induced up-regulation of the CHR-binding activity seen in wtEzrin transfected cells (FIG. 9C). That TNF-induced modulation of ezrin up-regulates CHR-binding activity was further evident from ezrin immunodepletion experiments. Endogenous ezrin was immunodepleted from control and TNF-treated nuclear extracts by treating extracts with anti-ezrin antibodies over 5 rounds. Immunodepleted extracts were then analyzed by EMSA for CHR-binding activity. As shown in FIG. 9D, depletion of ezrin from TNF-treated nuclear extracts completely abrogated TNF-induced up-regulation of CHR-binding activity observed in similarly treated but non-depleted nuclear extracts. Addition of recombinant ezrin back to the depleted TNF-extract significantly increased CHR-binding activity. These data apart from re-confirming ezrin-CHR interaction, also suggested that TNF-mediated inhibition of cyclin A transcription, at least partially, is mediated by TNF-induced ezrin modulations.

FIGS. 9A-D are explained in more detail as follows. (FIG. 9A) Nuclear extracts from quiescent (Q), serum stimulated (S) and serum stimulated and TNF treated (T) BAEC were incubated with biotinylated wild type or mutant CHR oligos. Protein-DNA complexes were captured on avidin agarose beads. Eluted proteins from beads were analyzed for ezrin protein in western blots. (FIG. 9B) Recombinant-ezrin or control DP1 proteins (1 μg) were incubated with CHR end-labeled oligonucleotide spanning CHR binding elements. For competition experiment indicated molar concentration of unlabeled oligos were added to reaction mix prior to the addition of radiolabeled probe. (FIG. 9C) Nuclear extracts from control and TNF-treated BAECs, transfected with wild type or dominant negative ezrin, were evaluated CHR binding activity in EMSAs. Over-expression of dominant negative ezrin significantly blunted TNF-induced upregulation of CHR binding activity. (FIG. 9D) Nuclear extracts from control (S) or TNF (T) treated cells were immunodepleted of ezrin by anti-ezrin antibodies. Depleted extracts alone or depleted extracts from TNF treated cells reconstituted with GST-ezrin were then analyzed for CHR binding activity. Autoradiographs shown are representative of at least 4 similar experiments.

### EXAMPLE 11

TNF modulates ezrin expression and sub-cellular distribution.

There are few if any accepted reports of nuclear expression and/or stimulus-induced nuclear translocation of full-length ERM proteins including that of ezrin. However, nuclear translocation of an alternatively spliced 55 kDa ezrin isoform has been reported earlier Kaul S. C. et al., Exp Cell Res. 1999;250:51-61. Since TNF treatment must translocate ezrin to the nucleus in order to bind cyclin A CHR elements in our system, the sub-cellular distribution of ezrin in control and TNF-treated EC employing a variety of experimental strategies was examined.

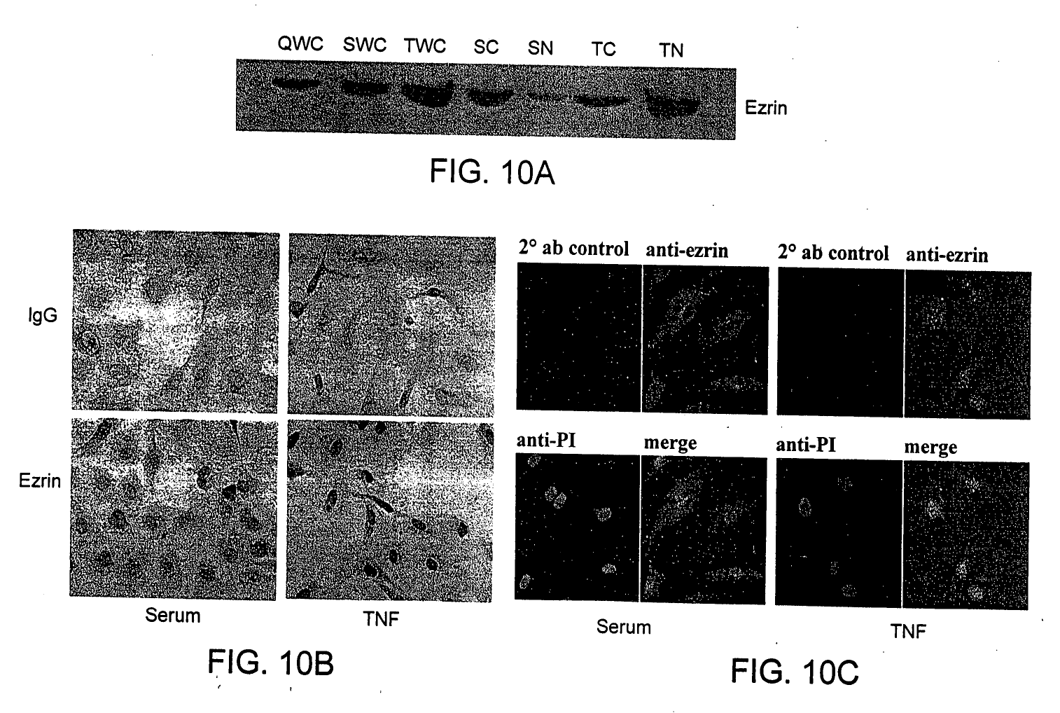

TNF treatment of EC for 24 h not only up-regulated ezrin expression in whole cell lysates but also substantially shifted the localization of ezrin predominantly to the nuclear extracts as opposed to predominantly in cytoplasmic extracts in the control cells (FIG. 10A). Cyto-immunochemical staining (FIG. 10B) of control and TNF-treated cells revealed a similar shift from cytoplasmic/peri-nuclear expression in control cells to predominantly nuclear expression in TNF-treated cells. This observation was further corroborated by laser confocal microscopy that revealed that, upon TNF-stimulation, ezrin co-localizes with propadium iodide stained nuclei (FIG. 10C). Exogenously transfected-ezrin showed a similar trend in response to TNF. In EC transiently transfected with wtEzrin expression vector tagged with VSVG protein, nuclear expression of the transgene was observed in response to TNF treatment. Taken together these data clearly demonstrate that TNF modulates the sub-cellular distribution of ezrin translocating it to the nucleus where it binds the target transcriptional repressor CHR-elements of the cyclin A promoter.

FIGS. 10A-C are explained more fully as follows. Whole cell (WC), cytoplasmic (C) and nuclear (N) extracts from quiescent (Q), serum alone stimulated (S), or TNF-treated (T) BAECs were analyzed for ezrin expression by western blots (FIG. 10A). (FIG. 10B) Representative cyto-immnochemical staining showing ezrin located predominantly in the cytoplasm and peri-nuclear region in serum stimulated cells and predominantly nuclear in TNF-treated cells. (FIG. 10C) Ezrin nuclear staining was independently confirmned by co-localization with propidium iodide in laser confocal microscopy experiments. Representative picture showing co-localization of ezrin with PI in TNF treated cells.

### EXAMPLE 12

Dominant Negative Ezrin Rescues EC from TNF-Mediated Inhibition.

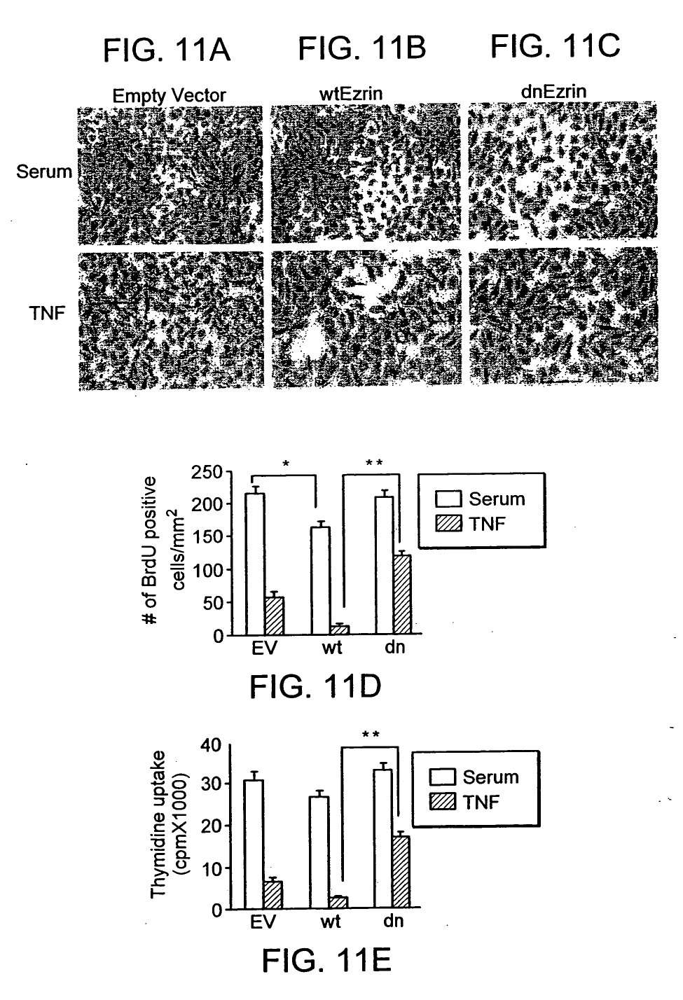

Having documented TNF-induced nuclear translocation and the ability of ezrin to bind to cyclin A CHR elements, the functional consequences of TNF-mediated ezrin modulation in EC was determined. To evaluate the effect of ezrin over-expression in the presence of TNF on cell proliferation, EC were transiently transfected with either wtEzrin or dnEzrin expression vectors. Cells were treated or not with TNF and BrdU (50 μg/mL) was added to all cultures for 6 hours. As shown in FIGS. 11A-C, in mock transfected cells (empty vector), TNF substantially reduced the number of BrdU positive cells compared to untreated cells (216±2.3/mm² in control vs 58±3.1/mm² in TNF-treated cells, p<0.01). Over-expression of wild-type ezrin not only reduced number of BrdU positive control cells (163±3.2/mm², p<0.05) but also exacerbated the effect of TNF on BrdU positive EC (1±1.6 vs 58±3.1/mm² in empty vector transfected cells, p<0.01). Interestingly however, over-expression of dominant negative ezrin significantly altered the number of BrdU positive cells under TNF treatment compared to those transfected with wild type ezrin under similar culture condition (117±3.4/mm² in dominant negative ezrin transfected vs. 11±1.6 mm² in wild-type transfected, p<0.001). Tritiated thymidine uptake assays were performed as an independent measure of cell proliferation in similarly transfected and treated cells and corroborated the findings of BrdU incorporation (FIG. 11C). These data provided first set of evidence that EC proliferation and therefore function may be improved by blocking the effect of TNF on ezrin modifications.

FIGS. 11A-E are explained in more detail as follows. Quiescent BAECs transiently transfected with empty vector, wild type and dominant negative ezrin were cultured in the absence (control) or presence of 20 ng/mL TNF for 24 hrs. Cells were labeled with BrdU (50 μg/mL) for last 6 hrs of culture. Cells were fixed and stained with anti-BrdU antibodies. BrdU positive cells were counted in at least 6 different visual fields. Representative cytoimmunochemical staining (FIGS. 11A-C) and average data from 3 similar experiments as bar graph (FIG. 11D) is shown. BAEC proliferation under similar conditions was confirmed independently by IH3-thymidine incorporation (FIG. 11E). * p<0.05; ** p<0.01.

### EXAMPLE 13

Ezrin Binds to Cyclin A Promoter In Vivo and Dominant Negative Ezrin Restores TNF-Mediated Down-Regulation of Cyclin A Promoter Activity and mRNA Expression.

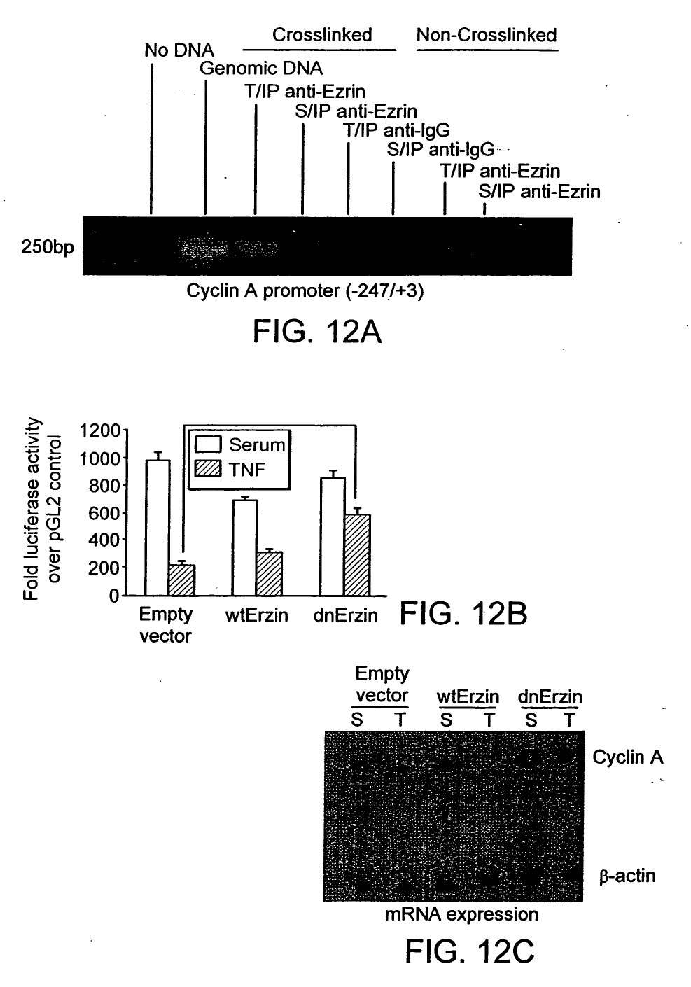

In vivo interaction of ezrin with Cyclin A-CHR elements was further confirmed by chromatin immunoprecipitation experiments. As shown in FIG. 12A, ezrin-chromatin complex was specifically immunoprecipated from TNF-treated cells, further corroborating ezrin-CHR binding. Since we have previously demonstrated that TNF acts by down-regulating cyclin A transcription resulting in EC cell cycle arrest Kishore R. et al., Circulation Res. 91 :307-14;2002), it was believed that improvement in EC proliferation by dominant negative ezrin, despite TNF exposure, reflects enhanced cyclin A promoter activity and mRNA expression. To address these questions, the effect of TNF on cyclin A promoter activity in ezrin expression vector transfected cells was examined. EC were co-transfected with wtEzrin or dnEzrin and a cyclin A promoter-reporter construct (cyclinA-luc −924/+100) and changes in luciferase activity in comparison with control luciferase vector (pGL2) transfected cells were determined. Promoter activity in empty vector transfected cells was significantly reduced in TNF treated cells (p<0.01) and was further reduced in cells transfected with wild type ezrin (FIG. 12B). Over-expression of dominant negative ezrin significantly blocked the negative effect of TNF on cyclin A promoter activity compared to that observed in wild type ezrin transfected cells (p<0.003). The effect of dominant negative ezrin on TNF-mediated cyclin A promoter activity was further corroborated by a significantly up-regulated cyclin A mRNA expression in dominant negative ezrin transfectants despite TNF treatment (FIG. 12C).

FIGS. 12A-C are explained in more detail as follows. (FIG. 12A) Representative PCR gel photograph showing amplification of 200 bp cyclin A promoter emcompassing CHR elements from ant-ezrin antibody immunoprecipitated chromatin. (FIG. 12B) BAECs co-transfected with cyclin A promoter-reporter construct and either wt or dnEzrin constructs and cultured in presence or absence of TNF for 24 h before were analyzed for luciferase activity. Bar graph represents average promoter activity from 3 similar experiments (*p<0.01). (FIG. 12C) Total RNA from empty vector wt or dnEzrin transfected cells under control or TNF-treatment conditions was evaluated for cyclin A mRNA expression by ribonuclease protection assays. Representative autoradiograph is shown.

### EXAMPLE 14

Transplantation of Dominant Negative Ezrin-Transfected HUVECs Improves Blood Flow Recovery in Nude Mice Hind-Limb Ischemia Injury.

To begin evaluating the effects of ezrin on EC function in vivo, we utilized the mouse hindlimb ischemia model See eg., one or more of the following publications for more details: U.S. Pat. Nos. 6,659,428; 5,980,887; EP1061800; WO 99/45775; 5,830,879; 6,258,787; 6,121,246; RE37,933, 5,851,521 and 5,106,386.

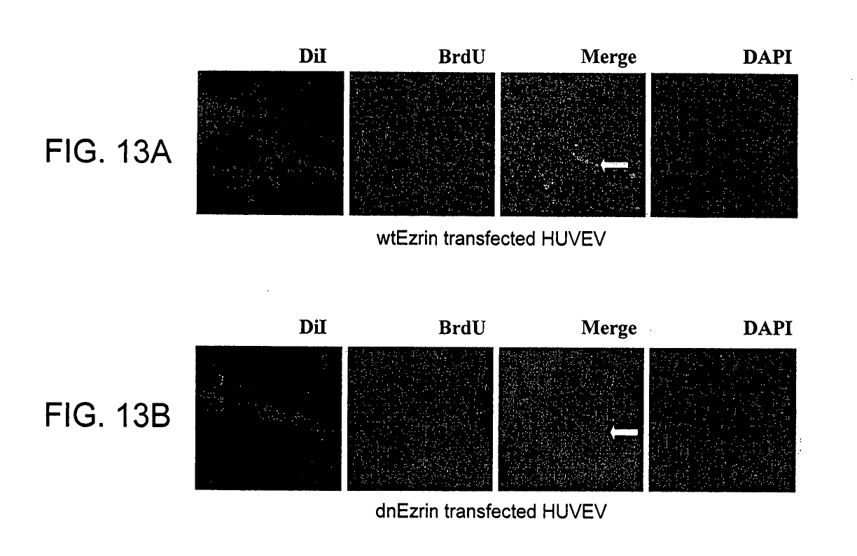

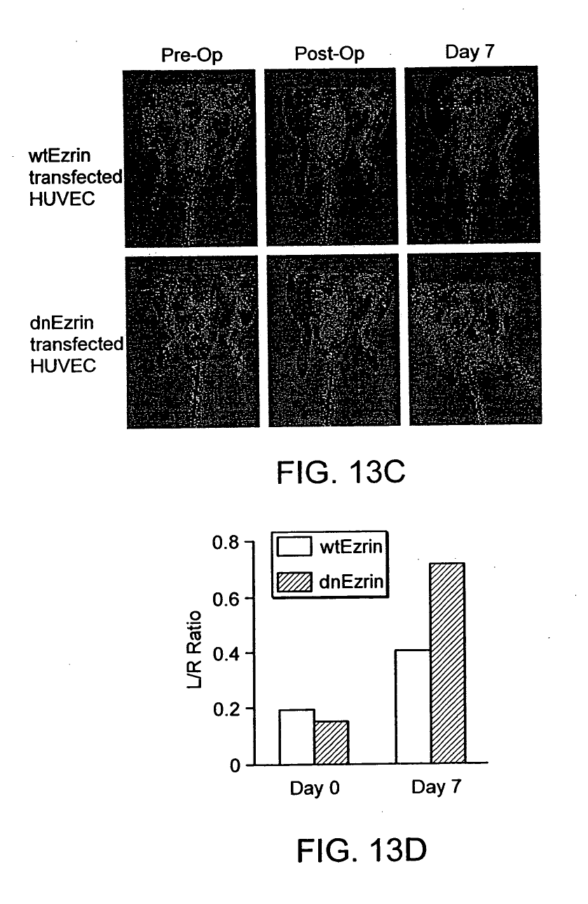

Hindlimb ischemia was established in nude mice and HUVEC transfected with wtEzrin or dnEzrin and labeled with DiI, were injected into the ischemic muscle along with the implantation of a BrdU micropump (Azlet). As shown in FIGS. 13A-B, immunofluorescent staining for double BrdU/DiI positive cells in the ischemic hindlimb tissue revealed a significantly higher number of proliferating EC in cells transfected with the dn vs. wtEzrin constructs. (dn=84±17/mm² vs. wt 28±13/mm², p<0.02. Cells were counted in eight randomly selected microscope fields from 2 randomly selected sections of tissue from each animal). In addition Laser Doppler imaging (FIGS. 13C and 13D) revealed that while hindlimb perfusion was equally reduced in both groups of animals immediately following surgical excision of the femoral artery, (Blue color denotes decreased blood flow in the operated limbs, white arrows in FIG. 13C and Black bars in FIG. 13D) at 7 days post-op the ratio of blood flow in the ischemic vs. non ischemic limb was improved in mice receiving dnEzrin transfected HUVECs compared with those receiving an equal number of transplanted EC transfected with wtEzrin (arrows in FIG. 13C, White bars in FIG. 13D). These data reveal that ezrin modulates EC proliferation in vivo, corroborating the present in vitro data, and also show that the increase in EC proliferation resulting from inhibition of ezrin function is associated with an increase in physiologic EC function. These data show that in vivo EC function is improved, in this case resulting in enhanced recovery of hindlimb perfusion, when ezrin function in impaired.

FIGS. 13A-D are explained in more detail as follows. HUVECs transfected with wild type or dominant negative ezrin and labeled with DiI, were injected into the ischemic muscle. A BrdU micropump (Azlet) was implanted at the time of surgery. (FIGS. 13A-B) Immunofluorescent staining for double BrdU/DiI positive cells in the ischemic hindlimb tissue revealed a significantly higher number of proliferating EC in cells transfected with the dn vs. wtEzrin constructs (dn=84±17/mm² vs. wt=28±13/mm², p<0.02). (FIG. 13C) Representative pictures of Laser Doppler imaging showing that at 7 days post-op the ratio of blood flow in the ischemic vs. non ischemic limb was improved in mice receiving dnEzrin transfected HUVECs compared with those receiving an equal number of transplanted EC transfected with wtEzrin. (FIG. 13D) Quantification of blood flow recovery data obtained from 6 mice in each group (Black bars=dnEzrin transfected HUVEC; white bars=wtEzrin transfected HUVEC).

### EXAMPLE 15

TNF-Induced Ezrin Modulations in EC are Mediated by RhoA Kinase Signaling.

Ezrin is known to be a target for tyrosine phosphorylation. Activation of ezrin via phosphorylation events is suggested to occur through RhoA signaling in some cell types. Accordingly, the role of RhoA kinase in TNF-induced functional changes in ezrin behavior was examined as follows.

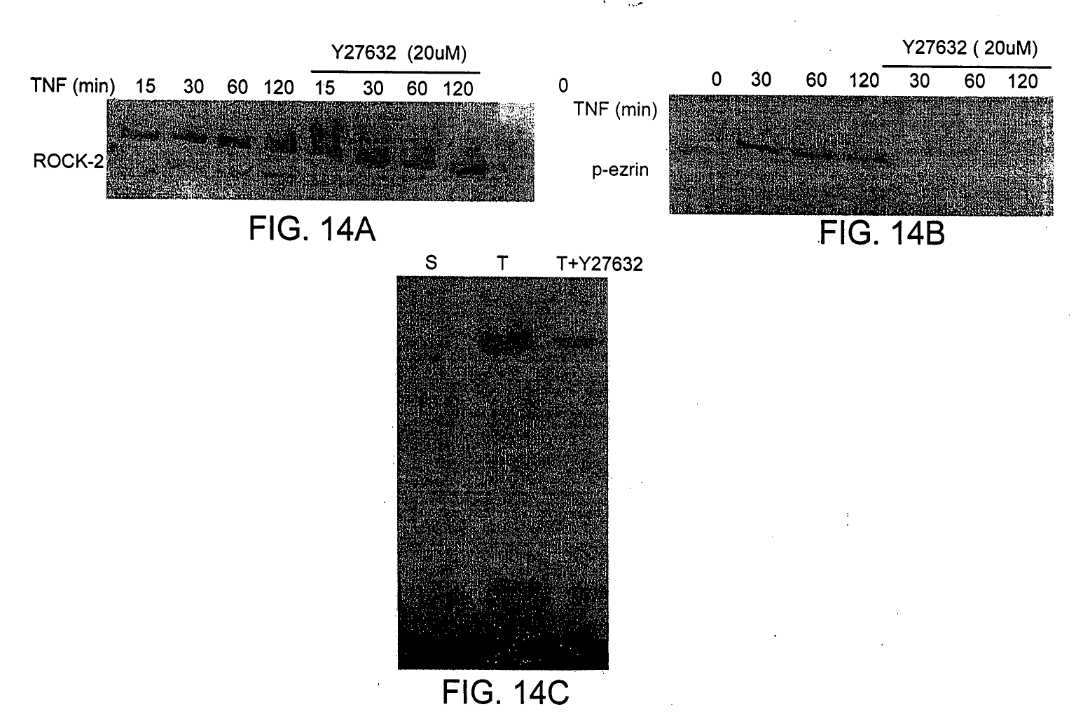

First, it was determined if TNF treatment of EC could induce the RhoA kinase (ROCK-2) expression. As shown in FIG. 14A, treatment of EC with TNF induced ROCK-2 within 15 minutes with persistent expression at all time points studied. Co-treatment of cells with ROCK-2 specific inhibitor Y27632 (20 micromolar ) at the time of TNF exposure completely abolished ROCK-2 induction, thereby confirming the specificity for ROCK-2 activation in response to TNF.

Next it was asked if TNF-treatment could phosphorylate ezrin and whether this phosphorylation is mediated via ROCK-2 activation. TNF-treatment of EC induced ezrin phosphorylation, which was completely inhibited by ROCK-2 inhibitor Y27632 at all experimental time points (FIG. 14B). To determine if inhibition of TNF-induced ROCK-2 activation could abrogate TNF-mediated upregulation of cyclin A-CHR binding activity, nuclear extracts from EC co-treated with TNF and Y27362 were analyzed for CHR binding activity. As indicated in FIG. 14C, Y27632 significantly reduced TNF-induced CHR binding activity, suggesting that ROCK-2 inhibition blocked ezrin phosphorylation and resultant nuclear translocation.

FIGS. 14A-C are explained in more detail as follows. Total cellular lysates from BAECs treated with TNF (20 ng/mL) in the presence or absence of specific Rho Kinase inhibitor Y27632 (20 μM) were analyzed in western blot for ROCK-2 (FIG. 14A) and phospho-ezrin (FIG. 14B) protein expression. (FIG. 14C) Serum (S), TNF (T) or TNF+Y27632 treated BAEC nuclear extracts were analyzed for CHR binding activity by EMSA assays. Representative autoradiograph from 3 experiments is shown.

### EXAMPLE 16

RhoA Kinase Inhibition Partially Attenuates TNF Inhibitory Effects on EC Proliferation.

To further confirm the involvement of RhoA signaling in TNF-mediated ezrin modulation and resultant EC dysfunction, additional functional experiments were performed as follows.

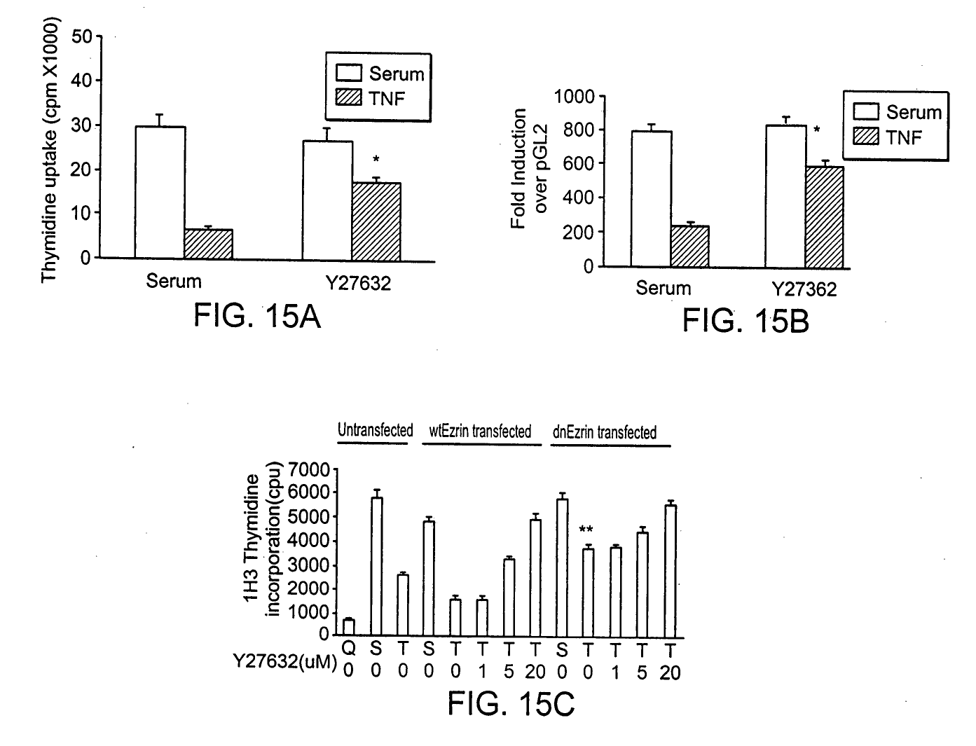

Treatment of EC with Y27632, partially abrogated the inhibitory effect of TNF on EC proliferation as evident by thymidine uptake assay (FIG. 15A). Co-treatment of EC with Y27632 in the presence of TNF had a similar positive effect on cyclin A promoter activity (FIG. 15B), as was observed in cells over-expressed with dominant negative ezrin. To provide further evidence that the improvement in EC function attained by inhibition of ROCK-2 was mediated by ezrin, we performed rescue experiments. EC transiently transfected with wtEzrin and treated with TNF were co-treated with increasing doses of Y27632 and cell proliferation was determined by thymidine incorporation. As shown in FIG. 15C, Y27632 dose dependently reversed ezrin/TNF mediated inhibition of EC proliferation further suggesting that ezrin is a downstream target of RhoA signaling in response to TNF treatment.

FIGS. 15A-C are explained in more detail as follows. Co-treatment of cells with Y27632 in the presence of TNF attenuates inhibitory effect of TNF on EC proliferation (FIG. 15A) and cyclin A promoter activity (FIG. 15B). Y27632 dose dependently reversed ezrin/TNF mediated inhibition in the EC proliferation. *p<0.01.

The following materials and methods were used in the above Examples unless otherwise mentioned.

1. Cell Culture: Bovine arterial endothelial cells (BAEC) were isolated as previously described¹¹. Cells in passages 3-6 were maintained in MEM supplemented with 10% FBS, 100U/mL streptomycin/penicillin and 50 μg/mL gentamycin in a 5% CO₂ atmosphere. For synchronization, cells were serum starved for 48-72 h.

2. Constructs, transient transfection and reporter assays: Cyclin A-Luc promoter reporter constructs, containing wild type and/or mutated fragments from mouse cyclin A promoter have been described before¹². BAECs were transiently transfected with luciferase reporter promoter constructs (pGL2-basic) containing various fragments representing either wt or mutated DNA binding sites from mouse cyclin A promoter using Lipofectamine(Gibco) following manufacturer's instructions. Cells were trypsinized, pooled and replated as per experimental requirement and were treated or not with 40 ng/mL recombinant human TNF for 24 h. Cells were harvested and assayed for luciferase activity using Berthold Lumat LB9501 luminometer. Luciferase activity was normalized to the alkaline phosphatase activity produced by a co-transfected alkaline phosphatase plasmid (pSVAPAP), which served as transfection efficiency control.

3. RNA isolation and ribonuclease protection assay (RPA): Synchronized BAEC were released from serum starvation and were further cultured in the presence or absence of TNF (40 ng/mL) for indicated time. Total cellular RNA was isolated using Trizol reagent (Life Technologies Inc.). For MRNA stability experiments, cells were treated with actinomycin D (Sigma, 5 μg/mL), before RNA was isolated. Human cyclin multiprobe DNA template (Pharmingen) and [α ³² P]UTP (NEN) was used to synthesize in vitro transcribed antisense riboprobe and RPAs were carried out using RPA III TM kit (Ambion) following manufacturer's instructions. Briefly, 5-10 μg of RNA was hybridized with radiolabeled probe and was then digested with RNase A/T1. RNase protected transcripts were then precipitated and were run on a 5% sequencing gels, dried and autoradiographed.

4. Nuclear run-on: Nuclear run-on experiments to measure nascent cyclin A transcripts were essentially performed as described elsewhere^{13, 14}.

5. Oligonucleotides, nuclear extracts and elctrophoretic mobility shift assay (EMSA): Following oligonucleotides representing CDE-CHR elements from cyclin A promoter (nucleotide −48/−16) were synthesized (MWG Biotech) and used in EMSA:

a) CDE-CHR (wt)-5′-CATTTCAATAGTCGCGGGATACTTGAACTGCA-3′,

b) mCDE-CHR-5′-CATTTCAATAGTCtaatGATACTTGAACTGCA-3′ and c) CDE-mCHR—5′-CATTTCAATAGTCCGCGGATACTgtccCTGCA-3′. mCDE-CHR and CDE-mCHR represents oligos wherein CDE and CHR sites were mutated (italics), respectively. Nuclear proteins isolation and EMSAs were carried out as described elsewhere¹⁵.

6. South-Western analysis: Nuclear proteins (50 μg) were resolved by 12% SDS-PAGE gels, electro-transferred to PVDF membrane and rocked for 10 min at 4° C. in binding buffer (25 mM Hepes/KOH, 60 mM KCI, lmM DTT, IlmM EDTA and 6M guanidinium chloride). The membrane was transferred to binding buffer containing 3M guanidinium chloride and incubated as in previous step. Renaturation was achieved by eight successive washes in binding buffer, each with a 50% stepwise reduction of guanidinium chloride, and a final 1 h incubation in the absence of guanidinium chloride plus 5% non-fat dry milk and 5 μg/mL sonicated salmon sperm DNA. The membrane was then incubated with 32P-labeled oligonucleotides in binding buffer plus 0.25% non-fat dry milk for 2-4 h at room temperature. Membranes were washed, air-dried and autoradiographed.

7. Uv cross-linking of DNA-protein complexes: The binding reactions were UV cross-linked by placing hand-held UV lamp ( 254 nm) at a distance of 2 cm from reactions for 20-30 min. UV cross-linked DNA-bound proteins were size fractionated on a 12% SDS-PAGE gel. Alternatively, following EMSA, wet gel was wrapped in saran wrap and auradiographed. The specific DNA-protein complex was excised from the gel UV cross-linked and incubated over-night with SDS sample buffer at room temperature. Eluted proteins were resolved by SDS-PAGE. Gel was dried and autoradiographed.

8. Statistical analysis. Data are presented as mean±SEM. Student's t test was used to evaluate the differences between groups and statistical significance was assigned when p≦0.05.

The Following Materials and Methods Were as Needed and Particularly With Respect to Examples 10-16:

9. Cell Culture: Bovine arterial endothelial cells (BAEC) and human umbilical cord endothelial cells (HUVEC) were isolated as previously described [Spyridopoulos, 1997 #2118]. Cells in passages 3-6 were maintained in MEM supplemented with 10% FBS, 100U/mL streptomycin/penicillin and 50 μg/mL gentamycin in a 5% CO₂ atmosphere. For synchronization, cells were serum starved for 48-72 h.

10. Recombinant protein, constructs, transient transfections and reporter assays: Recombinant human ezrin protein (GST-ezrin) and wild-type ezrin (pCB6-ezrin-VSVG) and dominant negative ezrin (pCB6-ezrin-Nter-VSVG) expression vectors were a kind gift from Dr. Monique Aprin and have been described before (Algrain M. et al., J Cell Biol. 1993;120:129-39). Cyclin A -Luc promoter reporter construct (cycA-Luc −924/+100), has been described before (Henglein B. et al., Proc NatlAcadSci. USA. 1994;91:5490-4, Kishore R. et al., Circulation Res. 91:307-14;2002).

For cyclin A promoter activity, BAECs were transiently transfected with luciferase reporter-cyclin A promoter construct (−924/+100) using Lipofectamine(Gibco) following manufacturer's instructions. Cells were trypsinized, pooled and replated as per experimental requirement and were treated or not with 40 ng/mL recombinant human TNF for 24 h. Cells were harvested and assayed for luciferase activity using Berthold Lumat LB9501 luminometer. Luciferase activity was normalized to the alkaline phosphatase activity produced by a co-transfected alkaline phosphatase plasmid (pSVAPAP), which served as transfection efficiency control. Ezrin expression vectors were similarly transfected. Transfection efficiency was monitored by irnmunostaining and western blot analysis of tagged VSVG protein and was comparable among transfections.

11. RNA isolation and ribonuclease protection assay (RPA):

Synchronized BAEC were released from serum starvation and were transfected with either empty vector or ezrin-expression vectors and were further cultured in the presence or absence of TNF (20 ng/mL) for indicated time. Total cellular RNA was isolated using Trizol reagent (Life Technologies Inc.). Human cyclin multiprobe DNA template (Pharmingen) and (α ³²P)UTP (NEN) was used to synthesize in vitro transcribed antisense riboprobe and RPAs were carried out using RPA III TM kit (Ambion) following manufacturer's instructions. Briefly, 5-10 μg of RNA was hybridized with radiolabeled probe and was then digested with RNase A/T1. RNase protected transcripts were then precipitated and were run on a 5% sequencing gels, dried and autoradiographed.

12. Oligonucleotides, nuclear extracts and elctrophoretic mobility shift assay (EMSA): The following oligonucleotides representing CHR elements from cyclin A promoter (nucleotide −31/−16) were synthesized (MWG Biotech) and used in EMSA: Wildtype CHR: 5′-ATACTTGAACTGCA-3′ and mutant CHR: 5′-ATACTgtccCTGCA-3′. Mutant CHR represents oligonucleotides wherein italicized nucleotides were substituted. Nuclear proteins isolation and EMSAs were carried out as described elsewhere (Kishore R. et al., Circulation Res. 91:307-14;2002).

13. South-Western analysis and Western blots: For modified South-western assays, nuclear proteins (50 μg) were reacted with biotin-wildtype and mutant CHR oligos in a scaled up EMSA reaction. Biotin-CHR-protein complexes were captured on ultralink streptavidin agarose beads (Pharmacia). Beads were washed eluted proteins were resolved by 12% SDS-PAGE gels, electro-transferred to PVDF membrane and rocked for 10 min at 4° C. in binding buffer (25mM Hepes/KOH, 60 mM KCl, 1 mM DTT, 1 mM EDTA and 6M guanidinium chloride). The membrane was transferred to binding buffer containing 3M guanidinium chloride and incubated as in previous step. Renaturation was achieved by eight successive washes in binding buffer, each with a 50% stepwise reduction of guanidinium chloride, and a final 1 h incubation in the absence of guanidinium chloride plus 5% non-fat dry milk and. The membrane was then incubated with anti-ezrin antibodies (Upstate Technologies) in binding buffer plus 0.25% non-fat dry milk for 2-4 h at room temperature. Membranes were washed, air-dried and autoradiographed. Western blots for ezrin, VSVG, phospho-ezrin and Rho Kinase were done using specific antibodies and standard protocols.

14. Immunocytochemical staining and Laser Confocal microscopy: For immunostaining, appropriately transfected and/or treated cells were fixed and endogenous peroxidase activity was blocked. Cells were then incubated with primary antibodies at 37° C. for 1 h or at 4° C. for overnight. Primary immune complex was detected with species matched biotinylated secondary antibodies and streptavidin peroxidase Krasinski K. et al., Circulation. 2001; 104:1754-1756, Asahara T. et al., Circulation. 1995;91:2793-801. For BrdU immunostaining, fixed cells were first incubated in 2M HCl for 10 min at 37° C. to denature DNA followed by blocking and primary antibody incubation steps. The slides were mounted with glycerol gelatin aqueous mounting media and examined on an Olympus Vanox-T microscope (Olympus American, Inc.,Melville, N.Y.). Pictures are recorded on Kodak Gold Plus films (Eastman Kodak Co,Rochester, N.Y.). Immunostaining for ezrin and phospho-ezrin was also evaluated by laser confocal microscopy as described elsewhere (Goukassain, 2003).

15. Chromatin immunoprecipitation (ChIP): ChIP assays were carried out using ChIP assay kit from Upstate Biotechnologies, NY, following manufacturer's instructions. Briefly, EC (3×10⁷/treatment group) were crosslinked by 1% formaldehyde at RT (22° C.) and lysed by sonication in 1 ml of ChIP IP buffer (0.1% SDS, 1% Triton X-100, 2 mM EDTA, 150 mM NaCl, 20 mM Tris—HCl, pH 8). Chromatin DNA was isolated from the lysates at this stage using gradient centrifugation. Total chromatin was digested using Sau3A1 enzyme before the IP step. Cross-linked DNA and proteins were purified from free uncross-linked proteins by centrifugation through a gradient of 5-8 M urea prepared in 10 mM Tris, pH 8.0, 1 mM EDTA. 0.6-1 Kb length chromatin was washed in a 10 mM Tris, pH 8.0, 1 mM EDTA buffer twice and purified by dialysis. Chromatin was then pre-cleared with 100 μg of salmon sperm DNA and 450 μl of protein A/protein A plus agarose (Santa Cruz Biotechnology) at 4° C. for 30 min followed by immunoprecipitations either with antibodies specific for ezrin and with a control antibody at 4° C. for 5 h. The precipitates were washed with ice-cold ChIP IP buffer and washing buffer (0.25 M LiCl, 1% Nonidet P-40, 1% deoxycholate, 1 mM EDTA, 10 mM Tris—HCl, pH 8). The DNA was extracted from the precipitates by incubation with 100 ρl of TE buffer (10 mM Tris—HCl at pH 8.0 and 1 mM EDTA), 2.5 μl of 10% SDS and 5 μg of proteinase K at 45° C. overnight. After 1 h incubation at 65° C. with an additional 2.5 ml of 5 M NaCl, the DNA fragments were purified with QiaEx II gel purification kit and subjected to PCR analysis using primers to amplify cyclin A promoter encompassing CHR elements. The PCR products were separated on 0.7-1% agarose gels and visualized by EtBr staining.

16. Hind limb ischemia, cell transplantation and laser Doppler imaging. Hindlimb ischemia was established in athymic nude mice (Harlan Labs) Yamaguchi, J. et al., Circulation,107:1322-8,2003 and HUVEC transfected with wtEzrin or dnEzrin and labeled with DiI, were injected into the ischemic muscle. A BrdU micropump (Azlet) was implanted at the time of surgery. Laser Doppler imaging to determine blood flow was performed immediately following surgery (day 0) and at day 7 following cell injections. Seven days after cell transplant the tissues were harvested and assayed for BrdU incorporation in the DiI labeled cell.

Taken together, the present examples show that TNF induced growth arrest of endothelial cells is mediated by transcriptional effects on cyclin A via binding activity of the CHR co-repressor elements in the cyclin A promoter. They also show that affinity purification and micro-sequencing experiments identified ezrin, a member of the cytoskeletal ERM family of proteins, as the transacting factor. Utilizing a series of biochemical, functional and signaling experiments, the examples provide early evidence that ezrin, in response to TNF, functions as a negative transcription factor and mediates cyclin A transcriptional repression in EC. Protein expression and immunostaining revealed that TNF facilitates ezrin translocation to the nucleus. The specificity of CHR-ezrin interactions in TNF-treated EC nuclear extracts was confirmed by documenting the binding of recombinant-ezrin protein to CHR-oligonucleotides in gel shift assays and by immunodepletion of ezrin from TNF-treated nuclear extracts which prevented TNF-mediated up-regulation of CHR binding activity. The functional role of ezrin modulation by TNF was evident by the over-expression of dominant-negative ezrin, which attenuated TNF-mediated suppression of EC proliferation, cyclin A promoter activity and mRNA expression. Furthermore, TNF-induced inhibition of neo-vasculogenesis by bone marrow derived endothelial progenitor cells in matrigel experiments in vivo, was reversed by the over-expression of dominant-negative ezrin. Finally, we show that the RhoA signaling pathway mediates TNF-induced modulation of ezrin function. TNF-induced RhoA kinase (ROCK) phosphorylated ezrin and a specific ROCK inhibitor Y27632 significantly blocked ezrin phosphorylation. Moreover pre-treatment of EC with Y27632 reversed TNF-mediated up-regulation of CHR binding activity and inhibition of cyclin A promoter activity and mRNA expression. The data provide the first evidence for a TNF-induced novel function of ezrin as a cyclin A transcription regulator. These findings disclose a novel fuinction for a cytoskeletal protein previously considered to participate only in extra-nuclear signaling pathways. Targeting ezrin activation directly or by modulating Rho kinase signaling represents a new strategy for modulating endothelial proliferation and angiogenesis.

All references disclosed herein are incorporated by reference. The following literature references (referred to by number in the disclosure) are specifically incorporated by reference.

1. Sato N, Goto T, Haranaka K, Satomi N, Nariuchi H, Mano-Hirano Y, Sawasaki Y. J Natl Cancer Inst. 1986;76:1113-21.

2. Rosenkranz-Weiss P, Sessa W C, Milstien S, Kaufman S, Watson C A, Pober J S. J Clin Invest. 1994;93 :2236-43.

3. Wang P, Ba Z F, Chaudry I H. Am J Physiol. 1994;266:H2535-41.

4. Kaartinen M, Penttila A, Kovanen P T. Circulation. 1996;94:2787-92.

5. Clausell N, Correa de Lima C, Molossi S, Liu P, Turley E, Gotlieb A l, Adelman A G, Rabinovitch M. British Heart Journal. 1995;73:534-539.

6. Hancock W W, Adams D H, Wyner L R, Sayegh M H, Karnovsky M J. Am J Pathol. 1994;14:1008-14.

7. Tanaka H, Sukhova G, Schwartz D, Libby P. Arterioscler Thromb Vasc Biol. 1996; 16:12-8.

8. Krasinski K, Spyridopoulos I, Kearney M, Losordo D W. Circulation. 2001;104:1754-1756.

9. Lopez-Marure R, Bernal A E, Zentella A. Biochem Biophys Res Commun. 1997;236:819-24.

10. Lopez-Marure R, Ventura J L, Sanchez L, Montano L F, Zentella A. Eur J Biochem. 2000;267:4325-33.

11. Spyridopoulos I, Sullivan A B, Kearney M, Isner J M, Losordo D W. Circulation. 1997;95:1505-1514.

12. Henglein B, Chenivesse X, Wang J, Eick D, Brechot C. Structure and cell cycle-regulated transcription of the human cyclin A gene. Proc Natl Acad Sci. USA. 1994;91:5490-4.

13. Sibinga N E, Wang H, Perrella M A, Endege W O, Patterson C, Yoshizumi M, Haber E, Lee M E. Interferon-gamma-mediated inhibition of cyclin A gene transcription is independent of individual cis-acting elements in the cyclin A promoter. J Biol Chem. 1999;274:12139-46.

14. Greenberg M E, Ziff E B. Stimulation of 3T3 cells induces transcription of the c-fos proto-oncogene. Nature. 1984;311:433-8.

15. Spyridopoulos I, Principe N, Krasinski K L, Xu S, Kearney M, Magner M, Isner J M, Losordo D W. Restoration of E2F expression rescues vascular endothelial cells from tumor necrosis factor-alpha-induced apoptosis. Circulation. 1998;98:2883-90.

16. Liu N, Lucibello F C, Komer K, Wolfraim L A, Zwicker J, Muller R. CDF-1, a novel E2F-unrelated factor, interacts with cell cycle-regulated repressor elements in multiple promoters. Nucleic Acids Res. 1997;25:4915-20.

17. Zwicker J, Lucibello F C, Wolfraim L A, Gross C, Truss M, Engeland K, Muller R. Cell cycle regulation of the cyclin A, cdc25C and cdc2 genes is based on a common mechanism of transcriptional repression. EMBO J. 1995;14:4514-22.

18. Liu N, Lucibello F C, Engeland K, Muller R. A new model of cell cycle-regulated transcription: repression of the cyclin A promoter by CDF-1 and anti-repression by E2F. Oncogene. 1998;16:2957-63.

19. Pines J. Cyclins and cyclin-dependent kinases: take your partners. Trends Biochem Sci. 1993;18:195-7.

20. Schulze A, Zerfass K, Spitkovsky D, Middendorp S, Berges J, Helin K, Jansen-Durr P, Henglein B. Cell cycle regulation of the cyclin A gene promoter is mediated by a variant E2F site. Proc Natl Acad Sci USA. 1995;92:11264-8.

21. Desdouets C, Matesic G, Molina C A, Foulkes N S, Sassone-Corsi P, Brechot C, Sobczak-Thepot J. Cell cycle regulation of cyclin A gene expression by the cyclic AMP-responsive transcription factors CREB and CREM. Mol Cell Biol .1995;15:3301-9.

22. Yoshizumi M, Wang H, Hsieh C M, Sibinga N E, Perrella M A, Lee M E. Down-regulation of the cyclin A promoter by transforming growth factor-betal is associated with a reduction in phosphorylated activating transcription factor-1 and cyclic AMP-responsive element-binding protein. J Biol Chem. 1997;272:22259-64.

23. Yu C, Takeda M, Soliven B. Regulation of cell cycle proteins by TNF-alpha and TGF-beta in cells of oligodendroglial lineage. J Neuroimmunol. 2000;108:2-10.

24. Beutler B. TNF, immunity and inflammatory disease: lessons of the past decade. J Investig Med. 1995;43:227-35.

25. Meldrum D R. Tumor necrosis factor in the heart. Am J Physiol. 1998;274:R577-95.

26. Modur V, Zimmerman G A, Prescott S M, McIntyre T M. J Biol Chem. 1996;271:13094-102.

27. Leibovich S J, Polverini P J, Shepard H M, Wiseman D M, Shively V, Nuseir N. Nature. 1987;329:630-2.

28. Pagano M, Pepperkok R, Verde F, Ansorge W, Draetta G. EMBO J 1992;11:961-71.

29. Wang W, Caldwell M C, Lin S, Fumeaux H, Gorospe M. EMBO J 2000;19:2340-50.

30. Liu N, Lucibello F C, Zwicker J, Engeland K, Muller R. Nucleic Acids Res .1996;24:2905-10.

31. Maity A, McKenna W G, Muschel R J. Cell Growth Differ. 1997;8:311-8.

32. Sachs A B. Cell. 1993;74:413-21.

33. Wilson G M, Brewer G. Prog Nucleic Acid Res Mol Biol. 1999;62:257-91.

34. Philips A, Chambeyron S, Lamb N, Vie A, Blanchard J. Oncogene. 1999;18:6222-32.

35. Goukassian D. A. et al., Circ Res, 93:162-69;2003.

The invention has been described in detail with reference to preferred embodiments thereof. However, it will be appreciated that those skilled in the art, upon consideration of this disclosure, may make modifications and improvements within the spirit and scope of the invention.

What is claimed is:

1. A method for modulating endothelial cell (EC) proliferation in a mammal, wherein the method comprises increasing or decreasing ezrin activity in the mammal by an amount sufficient to modulate proliferation of the cells.

2. The method of claim 1, wherein the ezrin activity is decreased by an amount sufficient to enhance the EC proliferation.

3. The method of claim 1, wherein the method further comprises administering to the mammal at least one ezrin modulating agent sufficient to decrease the ezrin activity and enhance the EC proliferation.

4. The method of claim 3, wherein the ezrin modulating agent is a nucleic acid or at least one amino acid sequence.

5. The method of claim 4, wherein the amino acid sequence is a competitor of Tumor Necrosis Factor-alpha (TNF-α).

6. The method of claim 5, wherein the competitor is at least one of TNF soluble receptor protein (TNFsr), a TNF antagonist, anti-TNF antibody; or an effective fragment or derivative thereof.

7. The method of claim 4, wherein the ezrin modulating agent is an anti-sense nucleic acid, anti-ezrin antibody, or an effective fragment or derivative thereof.

8. The method of claim 3, wherein the ezrin modulating agent reduces or blocks activity of Rho kinase (ROCK-2) in the endothelial cells.

9. The method of claim 8, wherein the ezrin modulating agent is Y27632.

10. The method of claim 4, wherein the ezrin modulating agent is a nucleic acid encoding a dominantly and negatively acting fragment of mammalian ezrin; or an effective fragment or derivative of the protein.

11. The method of claim 1, wherein the decrease in ezrin activity is at least about 50% as determined by a standard cyclin A promoter binding assay.

12. The method of claim 1, wherein the decrease in ezrin activity is at least about 50% as determined by a standard ezrin mRNA stability assay.

13. The method of claim 2, wherein the decrease in ezrin activity is associated with a decrease in ezrin tyrosine phosphorylation as determined by a standard protein phosphorylation assay.

14. The method of claim 1, wherein the ezrin activity is increased in an amount sufficient to decrease the EC proliferation.

15. The method of claim 14, wherein the ezrin modulating agent is tumor necrosis factor (TNF) or an effective fragment thereof.

16. The method of claim 14, wherein the EC proliferation is decreased by at least about 20% as determined by a standard restenosis assay.

17. A method for inducing formation of new blood vessels in a mammal, the method comprising decreasing ezrin activity in an amount sufficient to modulate formation of the new blood vessels in the mammal.

18. The method of claim 17, wherein the method further comprises administering to the mammal at least one ezrin modulating agent sufficient to decrease ezrin DNA binding activity relative to a control.

19. The method of claim 18, wherein the ezrin modulating agent is an inhibitor of Rho kinase (ROCK-2).

20. The method of claim 18, wherein the ezrin modulating agent is a nucleic acid encoding a dominantly and negatively acting fragment of mammalian ezrin; or an effective fragment or derivative of thereof.

21. The method of claim 18, wherein the method further comprises contacting endothelial cells (ECs) with the ezrin modulating agent sufficient to decrease ezrin activity therein.

22. The method of claim 21, wherein the method further comprises transforming endothelial cells with the ezrin modulating agent under conditions conducive to expressing the agent and administering the transformed cells to the mammal.

23. The method of claim 22, wherein the ezrin modulating agent is a nucleic acid encoding a dominantly and negatively acting fragment of mammalian ezrin; or an effective fragment or derivative thereof.

24. The method of claim 17, wherein the mammal has, is suspected of having, or will have ischemic tissue.

25. The method of claim 24, wherein the tissue is associated with an ischemic vascular disease.

26. The method of claim 1 or 17, wherein the method further comprises administering to the mammal at least one of an angiogenic protein, cytokine, hematopoietic protein; or an effective fragment thereof.

27. The method of claim 25, wherein the angiogenic protein is acidic fibroblast growth factor (aFGF), basic fibroblast growth factor (bFGF), vascular endothelial growth factor (VEGF-1), epidermal growth factor (EGF), transforming growth factor α and β (TGF-α and TFG-β), platelet-derived endothelial growth factor (PD-ECGF), platelet-derived growth factor (PDGF), tumor necrosis factor α (TNF-α), hepatocyte growth factor (HGF), insulin like growth factor (IGF), erythropoietin, colony stimulating factor (CSF), macrophage-CSF (M-CSF), angiopoetin-1 (Ang1) or nitric oxidesynthase (NOS).

28. The method of claim 26, wherein the hematopoietic factor is granulocyte-macrophage colony-stimulating factor (GM-CSF), VEGF, Steel factor (SLF, also known as Stem cell factor (SCF) ), stromal cell-derived factor (SDF-1), granulocyte-colony stimulating factor (G-CSF), HGF, Angiopoietin-1, Angiopoietin-2, M-CSF, b-FGF, and FLT-3 ligand.

29. The method of claim 28, wherein the protein is one of VEGF-B, VEGF-C, VEGF-2, VEGF-3; or an effective fragment thereof.

30. A method for reducing the severity of blood vessel damage in a mammal, wherein the method comprises decreasing ezrin activity in endothelial cells (EC) before, during or after a time in which the mammal is exposed to conditions conducive to damaging the blood vessels; wherein the decrease in ezrin activity is sufficient to reduce the severity of the blood vessel damage in the mammal.

31. The method of claim 30, wherein the method further comprises administering to the mammal at least one ezrin modulating agent to the mammal sufficient to decrease ezrin DNA binding activity relative to a control.

32. The method of claim 31, wherein the ezrin modulating agent is injected at or near the site of blood vessel damage in the mammal.

33. The method of claim 32, wherein the ezrin modulating agent is an inhibitor of Rho kinase (ROCK-2).

34. The method of claim 33, wherein the ezrin modulating agent is Y27632.

35. The method of claim 30, wherein the ezrin modulating agent is a nucleic acid encoding a dominantly and negatively acting mammalian ezrin protein; or an effective fragment or derivative of the protein.

36. The method of claim 31, wherein the blood vessel damage is restenosis associated with an invasive manipulation or associated with ischemia.

37. The method of claim 36, wherein the invasive manipulation is balloon angioplasty, or deployment of stent or catheter.

38. The method of claim 37, wherein the stent is an endovascular stent.

39. The method of claim 36, wherein the ischemia is associated with at least one of infection, trauma, graft rejection, cerebrovascular ischemia, renal ischemia, pulmonary ischemia, limb ischemia, ischemic cardiomyopathy, or myocardial ischemia.

40. The method of claim 30, wherein the ezrin modulating agent is administered to the mammal at least about 12 hours before exposing the mammal to the conditions conducive to damaging the blood vessels.

41. The method of claim 40, wherein the ezrin modulating agent is administered to the mammal between from about 1 to 10 days before exposing the mammal to the conditions conducive to damaging the blood vessels.

42. The method of claim 41, wherein the method further comprises administering the ezrin modulating agent to the mammal following the exposure to the conditions conducive to damaging the blood vessels.

43. A method for decreasing angiogenesis in a mammal, wherein the method comprises increasing ezrin activity in endothelial cells (ECs) of the mammal sufficient to decrease the angiogenesis.

44. The method of claim 43, wherein the method further comprises administering to the mammal at least one ezrin modulating agent sufficient to decrease ezrin DNA binding activity relative to a control.

45. The method of claim 44, wherein the ezrin modulating agent is injected at or near a site in which the decrease in angiogenesis is desired.

46. The method of claim 44, wherein the ezrin modulating agent is TNF Necrosis Factor alpha (TNF-α), Rho kinase; or an effective fragment or derivative thereof.

47. The method of claim 44, wherein the mammal has, is suspected of having, or is pre-disposed to develop cancer.

48. The method of claim 43, wherein the method further comprises administering at least one chemotherapeutic drug to the mammal.

49. A method for detecting a compound that modulates ezrin activity in the mammal, the method comprising the steps of:
1) introducing into cells a nucleic acid comprising at least part of a mammalian cyclin A gene linked to a detectable sequence,
2) adding at least one known or candidate ezrin modulating agent to the cells,
3) culturing the cells under conditions suited to expressing the nucleic acid and detecting the sequence in the presence and absence of the compound; and
4) determining the effect of the compound on the cells.

50. The method of claim 49, wherein step (4) of the method further comprises measuring at least one of proliferation and cycling of the cells.

51. The method of claim 49, wherein the nucleic acid used in the assay comprises a region spanning −1200 to +250 of the mammalian cyclin A gene.

52. The method of claim 51, wherein the nucleic acid comprises a region spanning −924 to +100 of the mammalian cyclin A gene.

53. The method of claim 52, wherein the nucleic acid comprises at least one of the AP1, ATF, and CDE-CMR promoter sites.

54. The method of claim 53, wherein the nucleic acid comprises the CDE-CMR promoter site between about −79 to about +100 of the mammalian cyclin A gene.

55. The method of claim 49, wherein the nucleic acid comprises the human cyclin A protein gene promoter spanning about positions −79 to about +100 of the gene which promoter is covalently linked in-frame to a sequence encoding a fluorescent or phosphorescent protein; or a detectable fragment thereof.

56. The method of claim 55, wherein the label is derived from a fluorescent jellyfish protein.

57. The method of claim 56, wherein the jellyfish protein is green fluorescent protein (GFP) or red fluorescent protein (RFP).

58. The method of claim 49, wherein the nucleic acid comprises the human cyclin A protein gene promoter spanning about positions −79 to about +100 of the gene which promoter is covalently linked in-frame to a sequence encoding the luciferase or beta-galactosidase enzyme; or a detectable fragment thereof.

59. A method for detecting a compound that modulates ezrin activity, the method comprising the steps of:
1) adding at least one known or candidate ezrin modulating agent to the cells,
2) culturing the cells under conditions suited to increase or decrease ezrin phosphorylation relative to a control; and
3) identifying an increase or decrease in ezrin phosphorylation relative to a suitable control as being indicative of the compound.

60. The method of claim 59, wherein step (3) of the method comprises performing an immunoassay.

61. The method of claim 60, wherein the immunoassay comprises performing a sandwich type immunoassay with an anti-phosphotyrosine antibody.

62. A method for detecting DNA binding between ezrin (or a DNA binding fragment thereof) and at least part of a mammalian cyclin A gene, the method comprising the steps of:
1) incubating at least part of a mammalian cyclin A gene with the ezrin protein or a DNA binding fragment thereof, wherein the incubation is conducted under conditions sufficient to form a specific binding pair between the cyclin A gene and the ezrin protein (or fragment),
2) adding at least one known or candidate ezrin modulating agent to the incubation medium; and
3) detecting presence of a specific binding pair between the cyclin A gene (or fragment) and the ezrin protein (or fragment) in the presence and absence of the compound, wherein a reduction or absence of the binding pair is taken to be indicative of a compound that reduces or blocks ezrin binding to the cyclin A gene.

63. The method of claim 62, wherein the cyclin A gene part is a detectably-labeled oligonucleotide comprising at least the CDE-CDR sequence.

64. The method of claim 63, wherein the detectable label is visualized by means of an automated or semi-automated fluorescence, colorimetric, or phosphorescence detection device.

65. The method of claim 63, wherein the specific binding pair is detected by performing an electrophoretic manipulation.

66. The method of claim 1, 17, or 30, wherein the method further comprises isolating endothelial progenitor cells (EPCs) from the mammal and contacting the EPCs with at least one of: an ezrin modulating agent, cytokine, angiogenic factor or hematopoetic factor.

67. The method of claim 66, wherein the method further comprises administering the EPCs to the mammal in an amount sufficient to modulate endothelial cell proliferation.

68. The method of claim 67, wherein the method further comprises administering at least one of the following to the mammal before, during of after administration of the EPCs: ezrin modulating agent, cytokine, angiogenic factor or hematopoetic factor.

69. A pharmaceutical product for inducing neovascularization in a mammal, wherein the product comprises endothelial cells, the product comprising at least one ezrin modulating agent, wherein cells formulated to be physiologically acceptable to a mammal.

70. The pharmaceutical product of claim 69, wherein the product is sterile and further comprises at least one angiogenic protein or nucleic acid encoding the protein.

71. The pharmaceutical product of claim 70, wherein the endothelial cells express the ezrin modulating agent.

72. The pharmaceutical product of claim 71, wherein the expression is transient.

73. A kit for the introduction of a endothelial cells into a mammal, the kit comprising at least one ezrin modulating agent and optionally at least one angiogenic or hematopoietic protein or nucleic acid encoding same, the kit further comprising a pharmacologically acceptable carrier solution, nucleic acid or mitogen, means for delivering the cells and directions for using the kit.

74. The kit of claim 73, wherein the means for delivering the endothelial cells is a stent, catheter or syringe.
